(19) United States
(12) Patent Application Publication
(10) Pub. No.: US 2004/0266993 A1
(43) Pub. Date: Dec. 30, 2004

(54) Non-immunoglobulin binding polypeptides

(76) Inventor: Glen A. Evans, San Marcos, CA (US)

Correspondence Address: PHILIP S. JOHNSON, JOHNSON & JOHNSON, ONE JOHNSON & JOHNSON PLAZA, NEW BRUNSWICK, NJ, 08933-7003, US

(21) Appl. No.: 10/611,655

(22) Filed: Jun. 30, 2003

(51) Int. Cl. C07K 16/44

(52) U.S. Cl. 530/387.3

(57) ABSTRACT

The invention provides a chimeric non-immunoglobulin binding polypeptide having an immunoglobulin-like domain containing scaffold having two or more solvent exposed loops containing a different CDR from a parent antibody inserted into each of said two or more loops and exhibiting selective binding activity toward a ligand bound by said parent antibody. Also provided is a chimeric non-immunoglobulin binding polypeptide having an immunoglobulin-like domain containing scaffold having less than about 20% sequence identity to a human immunoglobulin variable region framework domain, said immunoglobulin-like domain containing scaffold having two or more altered solvent exposed loops and exhibiting selective binding activity toward a disparate ligand. A chimeric ThyOx binding polypeptide having one or more altered immunoglobulin-like domain loop regions of a ThyOx family polypeptide and having selective binding activity toward a non-ThyOx ligand as well as a chimeric ThyOx carrier polypeptide comprising a at least one immunoglobulin-like domain containing scaffold derived from a ThyOx family polypeptide, and a heterologous binding polypeptide exhibiting selective binding activity toward a non-ThyOx ligand are further provided. Additionally, the invention provides nucleic acids encoding a non-immunoglobulin or ThyOx binding polypeptide of the invention.

### BACKGROUND OF THE INVENTION

This invention relates to the design and engineering of polypeptide binding molecules and, more specifically to design and production of non-immunoglobulin binding polypeptides having selective binding activity toward a predetermined molecule.

The war on fatal, debilitating and chronic diseases has entered the twenty-first century. Recent years have shown tremendous progress in the understanding of the development and progression of certain diseases. However, there has been only marginal decreases in death rates from most types of fatal diseases and the treatment of many debilitating and chronic diseases still has major hurdles to overcome become cures can be expected. For example, cancer remains a major fatal disease. Standard chemotherapy and radiation therapy generally involve treatment with therapeutic agents that impact not only the diseased cells but also other highly proliferative cells of the body, often leading to debilitating side effects. Therefore, it still remains desirable to identify therapeutic agents with a higher degree of specificity for the carcinogenic lesion. Similarly, therapeutic agents with greater specificity also are desirable to both increase efficacy and lower undesirable side effects.

The discovery of monoclonal antibodies (mAbs) in the 1970's provided great hope for the reality of creating therapeutic molecules with high specificity. Antibodies that bind to diseased cell antigens would provide specific targeting agents for therapy. However, while the development of monoclonal antibodies has provided a valuable diagnostic reagent, certain limitations restrict their use as therapeutic entities.

Because mAbs are usually developed in non-human species, one limitation encountered when attempts are made to use them as therapeutic agents is that they elicit an immune response in human patients. Chimeric antibodies join the variable region of the non-human species, which confers binding activity, to a human antibody constant region. However, the chimeric antibody often remains immunogenic and it is therefore necessary to further modify the variable region.

One modification is the grafting of complementarity-determining regions, (CDRs) which are impart part antigen binding onto a human antibody variable framework. However, this approach is imperfect because CDR grafting often diminishes the binding activity of the resulting humanized mAb. Other modifications that have been employed to reduce immunogenicity of a chimeric, humanized or non-human antibody include veneering and resurfacing of the antibody solvent exposed residues to remove T- and B-cell antigenic epitopes.

Attempts to regain binding activity or to remove antigenic epitopes on humanized or other types of modified antibodies require laborious, step-wise procedures which have been pursued essentially by a trial and error type of approach. For example, one difficulty in regaining binding affinity is because it is difficult to predict which framework residues serve a critical role in maintaining antigen binding affinity and specificity.

Combinatorial methods have been applied to restore binding affinity, however, these methods require sequential rounds of mutagenesis and affinity selection that can both be laborious and unpredictable. Consequently, while antibody humanization methods that rely on structural and homology data are used, the complexity that arises from the large number of framework residues potentially involved in binding activity has hindered rapid success.

Thus, there exists a need for therapeutic binding molecules that can exhibit similar or better binding characteristics as monoclonal antibodies. The present invention satisfies this need and provides related advantages as well.

### SUMMARY OF THE INVENTION

The invention provides a chimeric non-immunoglobulin binding polypeptide having an immunoglobulin-like domain containing scaffold having two or more solvent exposed loops containing a different CDR from a parent antibody inserted into each of said two or more loops and exhibiting selective binding activity toward a ligand bound by said parent antibody. Also provided is a chimeric non-immunoglobulin binding polypeptide having an immunoglobulin-like domain containing scaffold having less than about 20% sequence identity to a human immunoglobulin variable region framework domain, said immunoglobulin-like domain containing scaffold having two or more altered solvent exposed loops and exhibiting selective binding activity toward a disparate ligand. A chimeric ThyOx binding polypeptide having one or more altered immunoglobulin-like domain loop regions of a ThyOx family polypeptide and having selective binding activity toward a non-ThyOx ligand as well as a chimeric ThyOx carrier polypeptide comprising a at least one immunoglobulin-like domain containing scaffold derived from a ThyOx family polypeptide, and a heterologous binding polypeptide exhibiting selective binding activity toward a non-ThyOx ligand are further provided. Additionally, the invention provides nucleic acids encoding a non-immunoglobulin or ThyOx binding polypeptide of the invention.

### BRIEF DESCRIPTION OF THE DRAWINGS

FIG. 1 shows the design and sequence of a ThyOx family non-immunoglobulin binding polypeptide. FIG. 1A shows an amino acid alignment of Thy-1 and an antibody variable heavy chain region together with a deduced Ig-like domain containing scaffold consensus amino acid sequence. FIG. 1B shows the amino acid sequence of ThyOx non-immunoglobulin binding polypeptide containing CDR binding domains. FIG. 1C shows a diagram of the ThyOx non-immunoglobulin binding polypeptide.

FIG. 2 shows a schematic diagram (FIG. 2A), amino acid sequence (FIG. 2B), and the nucleotide sequence and corresponding amino acid sequence (FIG. 2C) of a chimeric ThyOx carrier polypeptide containing erythropoietin.

FIG. 3 shows the nucleotide and amino acid sequence of SuperEpo.

FIG. 4 shows a schematic diagram (FIG. 4A), amino acid sequence (FIG. 4B), and the nucleotide sequence and corresponding amino acid sequence (FIG. 4C) of a chimeric ThyOx carrier polypeptide containing glucagon-like peptide 1.

FIG. 5 shows a schematic diagram and the nucleotide sequence for the vector pEgea M3.

FIG. 6 shows a schematic diagram and the nucleotide sequence for the vector pEgea Q6.

### DETAILED DESCRIPTION OF THE INVENTION

The invention is directed to non-immunoglobulin binding polypeptides that exhibit selective binding activity toward a predetermined ligand. The non-immunoglobulin binding polypeptides are derived from an immunoglobulin-like domain containing scaffold that can be grafted with binding domains of a parent polypeptide to confer the binding specificity of the parent polypeptide onto the immunoglobulin-like domain containing scaffold. The non-immunoglobulin binding polypeptides of the invention have the advantages of being stable and modular in both the scaffold domain structures as well as in the ability to accept a broad range of heterologous polypeptide binding domains. Additionally, the immunoglobulin-like domain containing scaffolds can be readily obtainable from human sources so that their immunogenecity when used as a human therapeutic is negligible. The scaffolds of the invention also can be readily constructed to contain or omit naturally occurring polysaccharide chains or to include novel chains or other extra-scaffold moieties or polypeptide structures. Non-immunoglobulin therapeutic binding polypeptides of the invention can be rapidly and efficiently generated to increase the availability, specificity or efficacy of useful therapeutics for human diseases.

In one embodiment, the invention is directed to non-immunoglobulin binding polypeptides having antibody variable region complementarity determining regions (CDRs) inserted into a Thy1 immunoglobulin-like domain containing scaffold. The CDRs are inserted into the loop regions of the Thy1 polypeptide which allows the CDRs to fold into a similar confirmation as they would be in the three dimensional structure of the donor, or parent, antibody. The resulting hybrid, or chimeric, non-immunoglobulin binding polypeptide exhibits similar binding characteristics compared to the parent antibody.

In another embodiment, the invention is directed to a non-immunoglobulin binding polypeptide having altered immunoglobulin-like domain loops by amino acid substitution at some or all positions. The altered amino acid sequences in the loop domains confers selective binding activity toward a ligand other than that bound by the unaltered immunoglobulin-like domain containing scaffold. The amino acid alterations can be made at the nucleic acid or polypeptide level using a variety of methods known to those skilled in the art.

In yet another embodiment, the invention is directed to a non-immunoglobulin binding polypeptide derived from the ThyOx family of immunoglobulin-like domain containing polypeptides. The ThyOx polypeptides can be used as an immunoglobulin-like domain containing scaffold or as a carrier polypeptide to generate a binding polypeptide of the invention.

As used herein, the term “chimeric” when used in reference to a binding polypeptide of the invention is intended to mean a polypeptide composed of two or more heterologous polypeptides fused together into a single primary amino acid sequence. Joinder of two or more heterologous amino acid sequences can be performed by, for example, chemical, biochemical or recombinant means. A chimeric polypeptide can therefore include, for example, a recombinant fusion protein or a chemical conjugate as well as other molecular complexes well known to those skilled in the art. When used in reference to a non-immunoglobulin binding polypeptide or a ThyOx carrier polypeptide, the term is intended to refer to a polypeptide that is composed of two or more heterologous polypeptides where one heterologous portion corresponds to a non-immunoglobulin polypeptide. For example, a chimeric polypeptide can be composed of a scaffold domain from one molecule and a binding domain from a different molecule. Similarly, a chimeric polypeptide can be composed of a carrier domain from a ThyOx family polypeptide and a binding domain from a different molecule. Both portions of the chimeric polypeptide can be derived from the same or a different species. Various other examples of chimeric polypeptides are well known to those skilled in the art an are included within the meaning of the term as it is used herein.

As used herein, the term “non-immunoglobulin” when used in reference to a binding polypeptide of the invention is intended to mean that the non-immunoglobulin portion of the polypeptide is a molecule other than an antibody as they are produced by B cells. The term also is intended to exclude antibody fragments greater than complementarity determining regions or CDRs. Therefore, antibody variable region fragments greater than about 50, 75, 100 or 110 amino acids are not encompassed within the meaning of the term as it is used herein. Non-immunoglobulin binding polypeptides of the invention can include, for example, immunoglobulin-like domain containing polypeptides within all superfamilies and ThyOx family member polypeptides, which are specific members of the immunoglobulin-like domain containing superfamily polypeptides.

As used herein, the term “immunoglobulin-like domain” or “Ig-like domain” when used in reference to a scaffold is intended to refer to an art-recognized β-sandwich structural motif found in proteins of diverse function, including for example, extracellular matrix proteins, muscle proteins, immune proteins, cell-surface receptors and enzymes. Ig-like domain members have been divided into various superfamilies, including for example, the immunoglobulin, fibronectin type III and cadherin superfamilies. Other superfamilies containing the Ig-like domain structural motif include, for example, members of the PKD domain, β-galactosidase/glucuronidase domain, transglutamase two C-terminal domains, actinoxanthin-like, CuZn superoxide dismutase-like, CBD9-like, lamin A/C globular tail domain, clathrin adaptor appendage domain, integrin domains, PapD-like, purple acid phosphatase N-terminal domain, superoxide reductase-like, thiol:disulfide interchange protein DsbD N-terminal domain and invasin/intimin cell adhesion fragments superfamilies. Ig-like domain structural similarity is maintained between members of different superfamilies irrespective of significant sequence identity. The term is intended to include Ig-like domain members within and across each superfamily. Therefore, the term “immunoglobulin-like (Ig-like) domain containing superfamily” is intended to refer to an Ig-like domain containing member polypeptide within any of these superfamilies as well as others known in the art. A description of the different Ig-like domain containing superfamilies can be found, for example, in Clarke et al., Structure Fold. Des. 7:1145-53 (1999) and within structural databases such as at the URL pdb.weizmann.ac.il/scop/data/scop.b.c.b.html.

As used herein, the term “ThyOx” or “ThyOx family polypeptide” when used in reference to a non-immunoglobulin polypeptide of the invention is intended to mean a subclass of polypeptides within the immunoglobulin superfamily (IgSF) of immunoglobulin-like domain containing polypeptides that are related by their common β-sandwich structural motif and containing a scaffold framework structure similar to antibody variable region domains. Particular polypeptides within the ThyOx family of polypeptides include, for example, Thy-1, Ox2, GP40, Ox2-like protein and Ox2 homolog. Table 1 sets forth these exemplary immunoglobulin-like domain ThyOx polypeptides as well as their alternative nomenclatures used in the art.

TABLE 2
ThyOx Family of Polypeptides

| Name | Alternate | Ig domains | Structure | Function |
|---|---|---|---|---|
| Thy-1 | CD90, | 1 | VL | T cell, |
| | CDW90, K117 | | | brain |
| Ox2 | CD200 | 2 | VLCL | T cell, |
| | | | | brain |
| GP40 | CD7, leu-9, | | | T cell |
| | p41 | | | |
| Ox2-like | NP150572 | 2 | VLCL | Unknown |
| protein | | | | |
| Ox2 | NP042978 | 2 | VLCL | Unknown |
| homolog | | | | |

As used herein, the term “scaffold” is intended to mean a supporting polypeptide framework used to organize, orient and harbor heterologous binding domains or altered amino acid sequences conferring binding specificity to a ligand. A scaffold can be structurally separable from the amino acid sequences conferring binding specificity. The structurally separable portion of a scaffold can include a variety of different structural motifs including, for example, β-sandwich, β-sheet, α-helix, β-barrel, coil-coiled and other polypeptide secondary and tertiary structures well known in the art. A scaffold of the invention will also contain one or more regions that can be varied in amino acid sequence without substantially reducing the stability of the supporting framework structure. An exemplary region that can be varied includes a loop region segment that joins two strands of a β-sandwich or β-sheet.

Amino acid residues corresponding to the structurally separated portion of a scaffold is referred to herein as a scaffold framework. Immunoglobulin-like domain containing scaffolds of the invention exhibit less than about 50% amino acid identity to a human immunoglobulin variable heavy or light chain framework amino acid sequence. Generally, immunoglobulin-like domain containing scaffolds will exhibit, for example, amino acid sequence identity less than about 45%, about 40%, about 30%, about 20%, about 15% or about 10% compared to a human immunoglobulin variable heavy or light chain framework amino acid sequence. Residues of a scaffold that can be varied are referred to herein with reference to its structural properties such as a loop region or with reference to its ability to accommodate altered residues. Therefore, a scaffold region that can be varied is referred to as a scaffold variable region, mutable region, exchange region, alterable region or changeable region, for example. Residues conferring secondary or tertiary structural properties can be retained, modified or conserved so long as the overall structure of the scaffold is maintained. Those skilled in the art know, or can determine, which residues function in structural stability of a polypeptide scaffold as well as the extent to which such residues can be modified.

Specific examples of scaffolds of the invention include immunoglobulin-like domain containing superfamily members. These superfamily members contain a immunoglobulin-like domain characterized as a β-sandwich which can be used as a scaffold of the invention. The β-sandwich consists of about 80-150 amino acid residues containing two layers of antiparallel β-sheet in which the flat hydrophobic faces of the β-sheets pack against each other. Each β-sheet contains a loop region that can be varied in amino acid sequence so as to confer unique binding specificity onto the scaffold polypeptide. Examples of Ig-like domain containing superfamily members include, for example, ThyOx family member polypeptides as well as the various individual members within the immunoglobulin-like domain containing superfamilies described previously. Such individual members include, for example, T cell receptor, CD8, CD4, CD2, class I MHC, class II MHC, CD1, cytokine receptor, GCSF receptor, GMCSF receptor, hormone receptors, growth hormone receptor, erythropoietin receptor, interferon receptor, interferon gamma receptor, prolactin receptor, NCAM, VCAM, ICAM, N-caderin, E-caderin, fibronectin, tenascin, and I-set containing domain polypeptides, or a functional fragment thereof. Exemplary descriptions of these an other Ig-like domain containing superfamily members can be found in, for example, Isacke and Horton, The Adhesion Molecule FactsBook, Second Ed., Academic Press, San Diego (2000); Fitzgerald et al., The Cytokine FactsBook, Second Ed., Academic Press, San Diego (2001), and Marsh et al., The HLA FactsBook, Second Ed., Academic Press, San Diego (1999).

As used herein, the term “carrier” is intended to mean a supporting polypeptide framework that is attached to a binding polypeptide. In general, a carrier polypeptide is a scaffold polypeptide that is attached to a binding polypeptide in locations other than its framework variable regions. For example, a binding polypeptide can be attached to a carrier polypeptide at amino acid residues outside of its framework region or within its framework region. A carrier polypeptide can be fused to, for example, the amino-terminal, carboxyl-terminal or both termini of a binding polypeptide. Accordingly, a carrier polypeptide of the invention is a subset of a scaffold polypeptide of the invention.

As used herein, the term “complementarity determining region” or “CDR” is intended to mean a region containing one of three hypervariable loops (H1, H2 or H3) within the non-framework region of the immunoglobulin (Ig or antibody) V_{H} β-sheet framework, or a region containing one of three hypervariable loops (L1, L2 or L3) within the non-framework region of the antibody V_{L} β-sheet framework. Accordingly, CDRs are variable region sequences interspersed within the framework region sequences. CDR regions are well known to those skilled in the art and have been defined by, for example, Kabat as the regions of most hypervariability within the antibody variable (V) domains (Kabat et al., J. Biol. Chem. 252:6609-6616 (1977); Kabat, Adv. Prot. Chem. 32:1-75 (1978)). CDR region sequences also have been defined structurally by Chothia as those residues that are not part of the conserved β-sheet framework, and thus are able to adapt different conformations (Chothia and Lesk, J. Mol. Biol. 196:901-917 (1987)). Both terminologies are well recognized in the art. The positions of CDRs within a canonical antibody variable domain have been determined by comparison of numerous structures (Al-Lazikani et al., J. Mol. Biol. 273:927-948 (1997); Morea et al., Methods 20:267-279 (2000)). Because the number of residues within a loop varies in different antibodies, additional loop residues relative to the canonical positions are conventionally numbered with a, b, c and so forth next to the residue number in the canonical variable domain numbering scheme (Al-Lazikani et al., supra (1997)). Such nomenclature is similarly well known to those skilled in the art.

For example, CDRs defined according to either the Kabat (hypervariable) or Chothia (structural) designations, are set forth below in Table 2.

TABLE 2
CDR Definitions

| | Kabat¹ | Chothia² | Loop Location |
|---|---|---|---|
| V_{H} CDR1 | 31-35 | 26-32 | linking B and C strands |
| V_{H} CDR2 | 50-65 | 53-55 | linking C′ and C″ strands |
| V_{H} CDR3 | 95-102 | 96-101 | linking F and G strands |
| V_{L} CDR1 | 24-34 | 26-32 | linking B and C strands |
| V_{L} CDR2 | 50-56 | 50-52 | linking C′ and C″ strands |
| V_{L} CDR3 | 89-97 | 91-96 | linking F and G strands |

¹Residue numbering follows the nomenclature of Kabat et al., supra
²Residue numbering follows the nomenclature of Chothia et al., supra

¹ Residue numbering follows the nomenclature of Kabat et al., supra

² Residue numbering follows the nomenclature of Chothia et al., supra

The term “immunoglobulin variable region framework” or “immunoglobulin variable region framework domain” as it is used herein, is intended to mean the portion or portions of an antibody heavy or light chain variable region other than the CDRs. An antibody variable region framework will contain about four framework domains that correspond to the amino acid sequence that flank or intervene between the three CDR region sequences. For example, Framework (Fw) region 1 corresponds to amino acid residues amino terminal to CDR1. Framework region 2 corresponds to the amino acid residues separating CDRs 1 and 2. Similarly, framework region 3 corresponds to the amino acid residues separating CDRs 2 and 3 while framework region 4 corresponds to the amino acid residues carboxy terminal to CDR3. Immunoglobulin variable region frameworks are well known to those skilled in the art.

As used herein, the term “inserted” when used in reference to the incorporation of a CDR or other binding domain into a scaffold of the invention is intended to mean splicing of a CDR or other binding domain into a scaffold loop or variable region. Splicing results in the substitution of some or all of the scaffold variable region with some or all of the CDR or other binding domain amino acid residues. Insertion therefore can include the exact or partial exchange of non-scaffold framework amino acid residues with amino acids corresponding to a binding domain of a binding polypeptide. The substitution will confer some or all of the binding activity resident in the CDR or binding domain onto scaffold framework without substantial disruption of the framework structure. Insertion can be accomplished by various methods known to those skilled in the art including, for example, polypeptide synthesis, nucleic acid synthesis of an encoding amino acid as well by various forms of recombinant methods well known to those skilled in the art.

As used herein, the term “selective” when used in reference to non-immunoglobulin polypeptide binding activity is intended to mean that the polypeptide exhibits discriminating or preferential binding activity toward a target ligand compared to a non-target ligand. Therefore, a non-immunoglobulin binding polypeptide exhibiting selective binding activity will distinguish or recognize a target ligand preferentially over non-target ligands, other polypeptides or macromolecules. Preferential binding can be due to specificity, affinity, avidity, off rate, on rate or any combination thereof. Those skilled in the art will know, or can determine, preferential binding of a target ligand using binding methods well known to those skilled in the art.

As used herein, the term “functional fragment” when used in reference to a non-immunoglobulin polypeptide of the invention is intended to mean a portion of a non-immunoglobulin binding polypeptide which retains at least about the same ligand binding activity compared to the binding domain donor polypeptide or parent binding polypeptide or compared to the intact non-immunoglobulin binding polypeptide. Such functional fragments can include, for example, truncated, deleted or substituted amino acid residues of the immunoglobulin-like domain containing scaffold framework so long as it retains about the same binding activity as the donor, parent or intact binding polypeptide. An example of a functional fragment of a non-immunoglobulin binding polypeptide of the invention is a immunoglobulin-like domain polypeptide having its membrane anchoring domain removed. Binding activity can be retained, for example, where the three dimensional structure of the scaffold's supporting polypeptide framework is substantially retained.

Immunoglobulin-like domain containing scaffolds, non-immunoglobulin binding polypeptides and ThyOx family polypeptides of the invention as well as functional fragments thereof are intended to include polypeptides having minor modifications of a specified amino acid sequence but which exhibits at least about the same ligand binding activity as the referenced binding domain donor polypeptide, parent binding polypeptide or the intact non-immunoglobulin or ThyOx binding polypeptide. Minor modifications of polypeptides having at least about the same ligand binding activity as the referenced polypeptide include, for example, conservative substitutions of naturally occurring amino acids and as well as structural alterations which incorporate non-naturally occurring amino acids, amino acid analogs and functional mimetics.

For example, a Lysine (Lys) is considered to be a conservative substitution for the amino acid Arginine (Arg). Other conservative amino acid substitutions and functional equivalents are well know in the art and can be found described in, for example, in Lehninger Principles of Biochemistry, Nelson and Cox, Third Edition, 2000, Worth Publishers, New York and Biochemistry, Stryer, Fourth Edition, 1995, W. H. Freeman and Company, New York. Similarly, analog or mimetic structures substituting positive or negative charged or neutral amino acids, with organic structures having similar charge and spacial arrangements also is considered a functional equivalent of a referenced amino acid sequence so long as the polypeptide analog or mimetic exhibits at least about the same ligand binding activity as the referenced polypeptide. Given the teachings and guidance provided herein, those skilled in the art will known, or can determine, which mimetic structures will function as an equivalent of a non-immunoglobulin binding polypeptide or as a domain or amino acid residue thereof.

As used herein, the term “ligand” is intended to mean a molecule that can be bound by a binding polypeptide. It will be appreciated that the terms “ligand” and “ligand binding site” or “binding domain” are complimentary, and are herein described relative to each other. Thus, for pairs of interacting molecules such as antibody/antigen, receptor/ligand, enzyme/substrate and the like, either molecule can be the “ligand” or provide the “ligand binding site.”

Generally, a pair of interacting molecules will bind to each other with selective binding affinity or specificity.

A ligand to which a ligand binding site or binding domain makes contact can be any chemical or biological molecule of interest. For example, the ligand can be a naturally occurring macromolecule, such as a polypeptide, peptide, amino acid, nucleic acid, nucleotide, carbohydrate, lipid, or any combination thereof. A ligand can alternatively be a partially or completely synthetic derivative, analog or mimetic of such a macromolecule. Likewise, a ligand can be a small organic molecule, such as a naturally occurring molecule or a molecule prepared by conventional or combinatorial chemistry methods. Optionally, such as to facilitate detecting binding, a ligand can be modified by the addition of a detectable label or tag, or attached to a solid support, for example.

As used herein, the term “disparate ligand” is intended to mean a non-binding partner to a referenced immunoglobulin-like domain containing scaffold of a non-immunoglobulin binding polypeptide. Therefore, the term refers to a non-natural ligand to a scaffold polypeptide of the invention. A disparate ligand for a non-immunoglobulin binding polypeptide of the invention can correspond to, for example, the natural binding partner to a parent antibody, parent binding polypeptide or a molecule not known to bind to a scaffold polypeptide of the invention. Accordingly, the term as it is used herein refers to a ligand selectively bound by the binding activity conferred onto an immunoglobulin-like domain containing scaffold or ThyOx carrier polypeptide of the invention.

The invention provides a chimeric non-immunoglobulin binding polypeptide consisting of an immunoglobulin-like domain containing scaffold having two or more solvent exposed loops containing a different CDR from a parent antibody inserted into each of the two or more loops and exhibiting selective binding activity toward a ligand bound by the parent antibody.

Chimeric polypeptides of the invention consist generally of two or more heterologous amino acid sequences fused togther. More specifically, the two or more heterologous sequences confer different functions onto the resultant polypeptide. The donor functions can include, for example, an activity, a structural feature or any other property inherent in an amino acid sequence. For example, the chimeric non-immunoglobulin binding polypeptides of the invention can contain one or more amino acid sequences corresponding to a scaffold and one or more different amino acid sequences corresponding to a binding domain, or functional fragment thereof. Amino acid sequences corresponding to the scaffold portion confer, for example, structural functions while the amino acid sequences corresponding to the a binding domain confer binding activity. The sequences for either of the components of a chimeric binding polypeptide of the invention can be derived, for example, wholly or partially from other polypeptides or domains, generated de novo or a combination thereof.

A variety of approaches and avenues of construction can be employed to generate a chimeric polypeptide of the invention. The end result of any approach is the joinder of heterologous sequences that previously lacked an association within a primary amino acid sequence. The association or linkage of heterologous sequences can be generated by, for example, the insertion of an amino acid sequence of any size as a group as well as the substitution or change of a portion of a polypeptide into a different sequence.

Insertions, substitutions, changes, modifications and the like can be within a contiguous or non-contiguous stretch of amino acid residues within an acceptor polypeptide such as a scaffold or carrier polypeptide of the invention. For example, a chimeric polypeptide can be constructed through the alteration of a loop region of a immunoglobulin-like domain. The alteration can consist of, for example, changing all or some of the residues of the loop region. Moreover, changed residues can consist of a contiguous stretch of amino acids or they can consist of non-contiguous residues within the loop region. The changes can be made by, for example, insertion of one or more different polypeptide sequences or by changing selected residues to non-scaffold residues. Other approaches well known in the art can similarly be used so long as the resultant molecule is a chimeric polypeptide in as much as its structure consists of two or more heterologous sequences joined at the primary amino acid level.

A non-immunoglobulin portion of a non-immunoglobulin binding polypeptide can consist of any molecule other than an antibody or variable region functional fragment of an antibody that is greater than a CDR. Non-immunoglobulin binding polypeptides of the invention therefore exclude an antibody variable region framework. Polypeptides other than heavy or light chain variable region frameworks can be used as a non-immunoglobulin binding polypeptide of the invention.

A non-immunoglobulin binding polypeptide can contain additional polypeptide sequences, structures and moieties so long as there is at least one chimeric non-immunoglobulin binding polypeptide or at least one non-immunoglobulin portion other than an antibody variable region framework. For example, a non-immunoglobulin binding polypeptide can consist of a non-immunoglobulin binding portion and one or more domains or polypeptides that impart another function onto the non-immunoglobulin binding polypeptide. Accordingly, the non-immunoglobulin binding polypeptides of the invention can exhibit multiple functions, one of which, is binding to a desired ligand. Functions other than binding activity of the non-immunoglobulin binding polypeptide similarly can include, for example, an activity, a structural feature or any other property inherent in an amino acid sequence. Additionally, functions associated with other macromolecules, organic compounds or inorganic compounds similarly also can be imparted onto a non-immunoglobulin binding polypeptide of the invention by joinder of such molecules to the non-immunoglobulin binding polypeptide.

Multiple functions also can be conferred onto a non-immunoglobulin binding polypeptide through the construction of multimeric non-immunoglobulin binding polypeptide. As with the attachment or joinder of functional domains, polypeptides, macromolecules, or compounds to a non-immunoglobulin binding polypeptide to confer secondary characteristics onto a non-immunoglobulin binding polypeptide, two or more chimeric non-immunoglobulin binding polypeptides be attached or joined as components of a multimeric non-immunoglobulin binding polypeptide. For example, two or more non-immunoglobulin binding polypeptides can be joined in linear or branched form to produce a dimer, trimer or other multimer of chimeric non-immunoglobulin binding polypeptides. Monomers of the chimeric multimers can exhibit the same or different binding activities toward one or more ligands.

As described further below, approaches and methods for constructing chimeric non-immunoglobulin binding polypeptides are well known in the art. For example, approaches can include insertion, substitution or directed changes of amino acid sequences. Approaches for inclusion of secondary functional characteristics can include, for example, the fusion or attachment of functional domains, polypeptides or other moieties. Methods to implement such approaches can include, for example, recombinant construction and in vitro or in vivo synthesis, chemical synthesis, conjugation, linkers as well as the use of domains corresponding to affinity binding partners. Various other approaches and methods well known in the art can similarly be utilized to design and construct a chimeric non-immunoglobulin binding polypeptide of the invention.

Because the terms “donor” or “acceptor” reference, for example, the derivation or origination of a component sequence, or reference a relative orientation of a component sequence during construction of a chimeric non-immunoglobulin binding polypeptide, references to these terms herein are to be interpreted in the context of which they are used. In this regard, a non-immunoglobulin binding polypeptide of the invention can be described as having two donor polypeptides with components of each joined together to produce a chimeric polypeptide of the invention. Alternatively, a non-immunoglobulin binding polypeptide of the invention also can be described as having a donor polypeptide corresponding to one component of the chimeric polypeptide and an acceptor polypeptide corresponding to another component of the chimeric polypeptide. Either or both terms will be used for simplicity in understanding when describing a component of a parent polypeptide incorporated into a chimeric non-immunoglobulin binding polypeptide of the invention.

For example, chimeric non-immunoglobulin binding polypeptides can be viewed as having two donor parent molecules. One donor corresponding to the scaffold domain while the other donor polypeptide corresponding to a CDR containing antibody. Alternatively, where the binding domains are substituted in toto, the term donor can refer to the CDR containing antibody while the scaffold can be referenced as either a donor of the structural components of the resultant non-immunoglobulin binding polypeptide or it can be referenced as an acceptor of the inserted CDR domains. By analogy, where the binding domains are altered to model a predetermined binding domain by, for example, directed mutagenesis or selective synthesis changes, the donor polypeptide corresponds to the polypeptide harboring the predetermined binding domain and the scaffold can be referred to as either a donor or acceptor as described above. Alternatively, where a non-immunoglobulin binding polypeptide is generated through alteration of, for example, a loop region within an immunoglobulin-like domain containing scaffold, the donor of the resulting chimeric molecule generally refers to the parent scaffold polypeptide. For example, where a loop region is randomized followed by selection of a chimeric non-immunoglobulin binding polypeptide for a desired binding activity, a parent binding polypeptide donating predetermined binding domain sequences will be absent.

A chimeric non-immunoglobulin binding polypeptide is constructed from a scaffold polypeptide. A scaffold of the invention is a donor, or parent, polypeptide that confers structural characteristics onto the resultant non-immunoglobulin binding polypeptide. Donated structural characteristics include a secondary or tertiary conformation that stabilizes the orientation or conformation of a heterologous binding sequence. Stabilization of binding sequences occurs when there is sufficient structural integrity to impart selective binding activity of the chimeric non-immunoglobulin binding polypeptide toward a predetermined ligand.

Scaffold structures applicable for use in the binding polypeptides of the invention can consist of any of a variety of secondary or tertiary polypeptide structures known in the art. For example, scaffold polypeptide structures can include, for example, β-turn structures such as a β-sheet or β-barrel, an α-helix, coiled-coil, globin fold, α/β barrel, up and down barrel, Greek key motif, β-α-β motif, 4 helix bundle, α/β horseshoe domain, hydrolase fold and the like. Any of these structures have stable secondary structures and can be used as a scaffold to harbor one or more binding domains. Moreover, such structures can additionally be stabilized using amino acid bridges, selected amino acid sequences or synthetic linkers, for example. Such structures limit the rotational degrees of freedom about one or more atoms within the scaffold polypeptide and result in conformational restriction of the structure.

Amino acid bridges can be generated through, for example, disulfide bonds between cystine residues or through covalent bond formation between amino acid side chain residues. Amino acid bridges also can be generated by covalent bonds between amino- and carboxyl-terminal residues of the scaffold polypeptide. Non-covalent bonds also can be used to achieve a similar effect.

Restricted conformations also can be achieved by incorporating selected amino acids that restrict the rotational freedom. Proline is an example of an amino acid having a restrictive conformation. Incorporating this residue at one or more selected positions within a scaffold polypeptide will limit the number of possible conformations for the polypeptide backbone. Similar results can be achieved using non-amide bonds, such as carbon-carbon double bonds, for joining amino acid monomers of the polypeptide scaffold.

Chemical linkers, synthetic bridges and the like also can be used to limit the conformational freedom of a scaffold polypeptide of the invention. Such linkers can be chemically coupled or conjugated to a scaffold in one or more locations to restrict flexibility or rotation about an axis. The location for incorporation will depend on the selected scaffold structure but will generally be chosen, for example, to link two or more non-adjacent residues so as to lock one conformer in place. The locked scaffold conformation will maintain the heterologous binding domains in proper conformation or orientation to achieve selective binding activity of the resultant chimeric polypeptide.

Design approaches, methods and chemistry for incorporating restrictive amino acid conformations, amino acid analogs, mimetics, bridges, and synthetic linkers are well known in the art. Specific examples of amino acid analogs and mimetics that can be useful is such approaches can be found described in, for example, Roberts and Vellaccio, The Peptides: Analysis, Synthesis, Biology, Eds. Gross and Meinhofer, Vol. 5, p. 341, Academic Press, Inc., New York, N.Y. (1983). Other examples include peralkylated amino acids, particularly permethylated amino acids. See, for example, Combinatorial Chemistry, Eds. Wilson and Czarnik, Ch. 11, p. 235, John Wiley & Sons Inc., New York, N.Y. (1997). Yet other examples include amino acids whose amide portion (and, therefore, the amide backbone of the resulting peptide) has been replaced, for example, by a sugar ring, steroid, benzodiazepine or carbo cycle. See, for instance, Burger's Medicinal Chemistry and Drug Discovery, Ed. Manfred E. Wolff, Ch. 15, pp. 619-620, John Wiley & Sons Inc., New York, N.Y. (1995). Methods for synthesizing peptides, polypeptides, peptidomimetics and proteins are well known in the art (see, for example, U.S. Pat. Nos. 5,420,109; 5,849,690; 5,686,567; 5,990,273; in PCT publication WO 01/00656, and in M. Bodanzsky, Principles of Peptide Synthesis (1st ed. & 2d rev. ed.), Springer-Verlag, New York, N.Y. (1984 & 1993), see Chapter 7; Stewart and Young, Solid Phase Peptide Synthesis, (2d ed.), Pierce Chemical Co., Rockford, Ill. (1984).

One scaffold structure useful in the binding polypeptides of the invention consists of an immunoglobulin-like domain containing polypeptide. One advantage of immunoglobulin-like domain containing scaffolds is that they contain a backbone consisting of a β-sandwich which is inherently stable. Moreover, β-sandwich structures can tolerate a wide variety of change within each of the β-loops without substantially affecting the stability of the β-sandwich backbone. As with other scaffold structures, this scaffold polypeptide can be used alone or in combination with the methods and structures described herein that augment stabilization of the secondary or tertiary structure.

Immunoglobulin-like (Ig-like) domain containing scaffolds can be found in a wide variety of polypeptides ranging from extracellular matrix proteins, muscle proteins, immune proteins, cell-surface receptors and enzymes. Members of Ig-like domain containing polypeptides have been divided into various superfamilies. Three such superfamilies include the immunoglobulin, fibronectin type III and cadherin superfamilies. Other superfamilies containing the Ig-like domain structural motif include, for example, those described previously. Ig-like domain structural similarity is maintained between members of different superfamilies irrespective of significant sequence identity. Members within any of these superfamilies can be used as an immunoglobulin-like domain containing scaffold for the production of a non-immunoglobulin binding polypeptide of the invention. A description of the different Ig-like domain containing superfamilies can be found, for example, in Clarke et al., Structure Fold. Des. 7:1145-53 (1999) and within structural databases such as at the URL pdb.weizmann.ac.il/scop/data/scop.b.c.b.html.

Ig-like domain containing members of the immunoglobulin superfamily (IgSF) refers to a structural domain characterized as an anti-parallel, Greek-key, β-sandwich (Bork et al., J. Mol. Biol. 242:309-320 (1994)). In general, an IgSF Ig-like domain contains from about 7-10 β-strands of between 5-10 amino acids each, connected by loops of variable sizes, distributed between two antiparallel β sheets. The β-strands are conventionally designated A through G, based on the C1 domain structure, which is described further below. One sheet contains the E and B strands, and optionally A and/or D strands. The other sheet contains the G, F and C strands, and optionally an A or A′ strand in parallel with G, and/or a C′ and/or a C″ strand. A conserved disulfide bond between the B and F strands connects the two sheets in many Ig-like domain containing members of the IgSF.

The Ig-like domains of IgSF members have been further subdivided into either three subfamilies or four subfamilies. Briefly, Bork et al., supra, has delineated three subfamilies termed V, C1 and C2. Harpaz and Chothia, have added the subfamily I to this superfamily, J. Mol. Biol. 238:528-539 (1994). Subfamilies differ from each other by the number of strands of each of the β-sheets, and also by the degree of separation between the conserved cysteine residues that form a disulfide bridge between the two β-sheets.

For example, V-set or V-type Ig-like domain subfamily of the IGSF refers to an IgSF Ig-like domain that contains both a C′ and C″ strand between the C and D strands. A V-set Ig-like domain typically has a separation of between about 63 and 76 residues between the two Cys residues that form the conserved disulfide bond. Many leukocyte cell surface antigens contain IgSF Ig-like domains of the V-set family.

A C1-set or C1-type Ig-like domain refers to an IGSF Ig-like domain that contains a short C′ strand of about three residues, and has a separation of between 54 and 64 residues between the two Cys residues that form the conserved disulfide bond.

An I-set or I-type Ig-like domain consists of an IGSF Ig-like domain that contains an A′ strand and a short C′ strand of about three residues, and has a separation of between 44 and 51 residues between the two Cys residues that form the conserved disulfide bond.

C2-set or C2-type Ig-like domains refer to an IgSF Ig-like domain that lacks the D′ strand but contains a C′ strand, and has a separation of between 38 and 43 residues between the two Cys residues that form the conserved disulfide bond.

Specific examples of Ig-like domain containing members of the IgSF include for example, CD8, CD4, CD2, CD80, CD86, CTLA-4, T cell antigen receptor), HSV glycoprotein D, Junction adhesion molecule, Fc(IgG) receptor, class I MHC, and class II MHC, VCAM-1, ICAM-1, ICAM-2, and MADCAM-1. Additionally, members within the ThyOx family of polypeptides such as Thy-1, Ox2, GP40, Ox2-like protein and Ox2 homolog are included within the IgSF superfamily of immunoglobulin-like domain containing polypeptides. Numerous other members are well known to those skilled in the art and are explicitly included in the description herein.

Members of the cadherin superfamily contain Ig-like domains that are relatively conserved in comparison to the structure of Ig-like domains contained in the immunoglobulin superfamily. Specific examples of members within the cadherin superfamily include N-cadherin, E-cadherin (uromorulin) and C-cadherin ectodomain.

Similarly, members of the fibronectin superfamily contain Ig-like domains that are relatively conserved in comparison to the structure of Ig-like domains contained in the immunoglobulin superfamily. Specific examples of members within the FnIII superfamily include fibronectin, tenascin, neuroglian, integrin β₄ subunit, growth hormone receptor, erythropoetin receptor, prolactin receptor, IL-4 receptor α chain, GC-SF receptor, INF-8 receptor α chain, and IL-10 receptor.

Ig-like domain containing members of superfamilies other than those within the immunoglobulin, cadherin or fibronectin type III superfamilies also can be used as a non-immunoglobulin scaffold polypeptide of the invention. Examples of these superfamilies include those containing a PKD domain such as Polycystein-1; β-galactosidase/glucuronidase domain such as β galactosidase and β glucuronidase; transglutamase two C-terminal domains such as human coagulation factor XIII and human TGase; actinoxanthin-like such as macromycin, neocarz ino-statin, actinoxanthin and kedarcidin; CuZn superoxide dismutase-like such as Cu, Zn superoxide dismutase and copper chaperone for superoxide dismutase; CBD9-like such as CBD9 and cellobiose dehydrogenase; lamin A/C globular tail domain such as laminin A/C; clathrin adaptor appendage domain such as α-adaptin ear domain; integrin domains such as domains of integrin α; PapD-like such as pilus chaperone PapD; purple acid phosphatase N-terminal domain; superoxide reductase-like such as superoxide reductase and desulfoferro doxin C-terminal domain; thiol:disulfide interchange protein DsbD N-terminal domain; and invasin/intimin cell adhesion fragments such as invasin and intimin.

Ig-like domain containing members of superfamilies other than those within the immunoglobulin, cadherin or fibronectin type III superfamilies also can be used as a non-immunoglobulin scaffold polypeptide of the invention.

Immunoglobulin-like domain containing scaffold polypeptides can be converted into chimeric non-immunoglobulin binding polypeptides of the invention using a variety of approaches described herein. One approach consists of inserting into one or more loops between anti-parallel β-strands forming a β-sandwich of an Ig-like domain one or more binding domains from a donor, or parent, binding polypeptide.

Any parent polypeptide can be used as a donor of binding domains that has characterized regions participating in selective binding to a ligand. The regions can be delineated as an intact or independently stable domain that maintains binding activity when isolated from its parent polypeptide. Regions also can be delineated as contiguous amino acid residues that require, in whole or in part, conformational stabilization from secondary or tertiary structure of its parent polypeptide. Alternatively, binding domain regions can consist of non-contiguous amino acid residues that when brought together in three-dimensional space together form an active binding domain of a parent polypeptide. Additionally, binding domain regions also can consist of specific amino acid residues contained within an active site of a parent polypeptide.

These and other parent polypeptide binding domains can be, for example, excised and placed into an Ig-like domain containing scaffold structure of the invention to confer binding characteristics of the parent polypeptide onto an Ig-like domain containing scaffold of the invention. Similarly, these and other parent binding polypeptide binding domains can be, for example, duplicated or reproduced within an Ig-like domain containing scaffold of the invention to confer binding characteristics of the parent polypeptide onto a scaffold of the invention.

The level of characterization of the parent polypeptide binding domain can influence the method used to reproduce a donor binding domain on an Ig-like domain containing scaffold of the invention. For example, when the parent polypeptide binding domain is intact, contiguous, or less characterized it can be beneficial to replicate a large portion, including the entire binding domain, onto a scaffold polypeptide of the invention. Replicating large portions of the binding domain will ensure transfer of the active binding residues to the scaffold polypeptide. In contrast, where the binding domains are non-contiguous or more characterized, it can be beneficial to transfer only those residues or sections of amino acid residues participating in binding to a scaffold of the invention. Participating residues can include, for example, amino acid residues that contact a ligand and lower the free energy of binding as well as those residues that orient or contribute to conformation of the contacting residues.

One or more binding domains described above, as well as others well known to those skilled in the art, can be inserted into a loop of a β-sandwich contained within an Ig-like domain containing scaffold of the invention. Following the teachings and guidance provided herein, a variety of formats and structures of chimeric non-immunoglobulin binding polypeptides can be readily generated by combining one or more binding domains from a parent polypeptide and a Ig-like domain containing scaffold. For example, non-immunoglobulin binding polypeptides can be generated that contain a single donor binding domain which confers the binding activity of the parent binding polypeptide, multiple donor binding domains that together confer the binding activity of the parent binding polypeptide, multiple donor binding domains that individually confer a different binding activity from one or more parent binding polypeptides or multiple donor binding domains where sets of two or more donor domains together constitute and confer a different binding activity of one or more parent polypeptides.

Other formats and structures similarly can be generated using the teachings and guidance provided herein. Those skilled in the art will understand that various combinations of the binding domain formats and binding polypeptide structures such as those described above, for example, can be generated from the insertion of one or more donor binding domains into a structure-independent region of an Ig-like domain containing scaffold to produce a chimeric binding polypeptide having a desired binding specificity or specificities. Therefore, chimeric non-immunoglobulin binding polypeptides of the invention can be readily generated that include, for example, monospecific, bispecific, trispecific or higher order multispecific binding activities.

Selection of compatible donor binding domains and Ig-like domain containing scaffolds will depend, for example, on the number of donor binding domains and the secondary or tertiary structure of the Ig-like domain containing scaffold. For example, where it is desirable to incorporate a single binding domain from a parent binding polypeptide the spacing and number of anti-parallel β-sheets within a β-sandwich is less important then where it is desirable to incorporate multiple binding domains that constitute an active binding domain when brought together in three-dimensional space. Examples of the former include the incorporation of an intact receptor or a receptor binding domain of a ligand into a loop region of a β-sheet. Examples of the latter include the incorporation of three antibody variable region CDRs into spatially adjacent loop regions to mimic an antibody variable region antigen binding pocket or the incorporation of both heavy and light chain variable region CDRs into six spatially adjacent loop regions to generate a functional mimic of the complete binding pocket of a dimeric antibody variable region.

Because an Ig-like domain is structurally conserved between members of the Ig-like domain superfamily, structural compatibility will exist between parent binding polypeptides and Ig-like domain containing scaffolds. Accordingly, functional compatibility also will exist when incorporating multiple binding domains such as antibody CDRs, for example, such that binding activity of the parent antibody also will be retained in the resultant chimeric non-immunoglobulin binding polypeptide. Therefore, when selecting donor binding domains from a parent polypeptide within a structurally related family such as a scaffold polypeptide, construction of the resultant chimeric non-immunoglobulin binding polypeptide will generally proceed by replacement of an analogous non-structural region in the scaffold polypeptide with one or more corresponding regions constituting a binding domain within the parent polypeptide.

Antibodies constitute exemplary parent binding polypeptides because, for example, they are prevalent, exist with a broad range of binding specificities or can be generated with relative ease. Antibody structure as well as their binding domains, or CDRS, are well characterized and can be routinely manipulated. Given the teachings and guidance provided herein, those skilled in the art can obtain the nucleotide or amino acid sequence of antibody variable region heavy and light chains, identify the CDR responsible for binding activity and insert one or more CDRs into an Ig-like domain containing scaffold to produce a chimeric binding polypeptide of the invention.

Briefly, antibodies are bivalent multichain proteins containing two pairs of light chains of either κ or λ isotype, and two pairs of heavy chains of γ, ε, δ, α or μ isotype. Both chains are composed of multiple repeats of Ig-like domains of about 100 amino acids, with central Cys residues separated by about 70 residues forming a disulfide bridge. The light chain is formed by two of these domains, called the variable (V_{L}) and the constant (C_{L}) domain. The heavy chain is formed by a variable domain (V_{H}) and either three or four constant domains (CH₁, CH₂, CH₃, CH₄). One heavy and one light chain combine to form one of the antigen binding sites within bivalent tetramer.

The antigen binding sites of antibodies are formed primarily by six loops, known as “complementarity determining regions,” or “CDRs.” Three loops are donated by each of the variable heavy and light chains. The entire antigen binding activity is therefore contained within the variable region fragments or Fᵥ, or V_{H}-V_{L} dimer. As described previously, CDRs can be described by their characteristic sequence hypervariability or by structural variability, Kabat et al., supra, and Chothia and Lesk, supra, respectfully. The positions of CDR within a canonical antibody variable region domain have been determined and described previously, Al-Lazikani et al., supra; Morea et al., supra, and are set forth in Table 2 as they are defined according to either a hypervariable or structural designations.

With regard to the three-dimensional structure of antibodies and each of their corresponding CDRs, within the heavy chain variable region V_{H} CDR1 packs across the top of the two β sheets, and contains V_{H} residues numbered 26 to 32 according to the Chothia numbering scheme. Variations in the size of the V_{H} CDR1 can be due to insertions at position 31, for example. V_{H} CDR2 is located between the C′ and C″ strands, and contains Chothia residues numbered 52 to 56. The V_{H} CDR3 is located in the hairpin loop linking the F and G strands, and contains Chothia residues numbered 95 to 102. Variations in the size of the V_{H} CDR3 can be due to insertions at position 100, for example.

A similar structural arrangement of CDRs is found within an antibody light chain variable region. For example, V_{L} CDR1 also packs across the top of the two β sheets, and contains V_{L} residues numbered 26 to 32 (Vκ) or 25 to 32 (Vλ) according to the Chothia numbering scheme. Variations in the size of the V_{L} CDR1 can be due to insertions between positions 30 and 32, for example. V_{L} CDR2 is located in the hairpin loop linking the C′ and C″ strands, and contains Chothia residues numbered 50 to 52. Finally, V_{L} CDR3 is located in the hairpin loop linking the F and G strands, and contains Chothia residues numbered 91 to 96.

The identification, excision, grafting or manipulation of antibody CDRs and their related variable region framework sequences are well known in the art. Similarly, the methods of CDR grafting, antibody humanization and other methods of modifying antibody molecules are well known in the art. All of such methods can be applied by analogy to the chimeric non-immunoglobulin binding polypeptides of the invention given the teachings and guidance provided herein. Such methods can be found described in, for example, U.S. Pat. Nos. 5,585,089, 5,693,762, 6,180,370, 5,693,761, 5,225,539, 5,565,332, 5,859,205, 6,054,297, directed to humanization or CDR grafting; U.S. Pat. No. 5,712,120, and publications WO 98/52976, WO 00/34317, directed to de-immunization; publication EP-A-0519596, directed to veneering, U.S. Pat. Nos. 5,639,641, 5,766,886, directed to resurfacing; U.S. Pat. Nos. 4,946,778, 5,260,203, 6,207,804, 5,091,513, directed to single chain antibodies, and U.S. Pat. No. 5,910,573, directed to designer antibodies.

Insertion of CDRs or other binding domains into an Ig-like domain containing scaffold can be performed by, for example, splicing, substituting, or altering the existing loop sequence to correspond to the parent polypeptide binding domain. For example, CDRs can be spliced in frame within a loop of an Ig-like domain containing scaffold to generate an extension in the loop corresponding to the inserted binding domain. Substitution of CDRs can be accomplished by, for example, removing non-structural residues of a loop region corresponding to the size of a CDR to be inserted. Alternatively, specific residues within a loop region can be changed to generate a sequence identical or substantially similar to a donor binding domain. These and other modes of inserting a binding domain into an acceptor polypeptide can be applied to Ig-like domain containing scaffolds as well as to other structural categories of scaffold polypeptides of the invention.

In some instances, it can be desirable to modify the length of the loop region to further model the spacing of binding domains within the parent binding polypeptide. Additionally, in the chimeric non-immunoglobulin binding polypeptides of the invention, one or several additional residues N-terminal, C-terminal or both amino- and carboxyl-terminal to structurally or sequence defined antibody CDR loop residues can be optionally included. Example I below exemplifies the insertion of antibody variable region CDRs into a Thy-1 Ig-like domain containing scaffold. Briefly, a sequence alignment was performed and residues deleted within the Thyl scaffold to more closely mimic the CDR spacing found in an antibody molecule. In this regard, the three loop regions of the β-sheet structures within Thy1 correspond to amino acid residue positions 47-51, 67-98 and 130-140. The amino acid sequences of heavy chain CDRs from a parent antibody were substituted into these positions by chemical synthesis of the encoding polynucleotide. Thus, Thy1 amino acids corresponding to three different loop regions were replaced by codon sequences encoding CDRs 1, 2 and 3, respectively. Additionally, amino acid residues 81-96 of the Thy1 sequence were deleted to spatially align the inserted CDRs with its parent antibody variable region sequence. The encoding nucleic acid was introduced into host cells and expressed recombinantly to produce the chimeric non-immunoglobulin binding polypeptide having binding specificity for the ligand of the parent antibody.

The example described above employing Thy1 as an immunoglobulin-like domain containing scaffold is directly applicable to the other superfamily members of Ig-like domain containing polypeptides described herein or well known to those skilled in the art. Using the teachings and guidance provided herein, it is a routine application to insert CDRs or other binding domains from a parent binding polypeptide into an Ig-like domain containing scaffold to produce a chimeric non-immunoglobulin binding polypeptide of the invention. For example, a CDR or all three CDRs from a antibody variable region can be inserted into a single loop region or different loop regions, respectively, of an Ig-like domain containing scaffold to produce a non-immunoglobulin binding polypeptide. Insertion of a single CDR or parent binding domain will produce a non-immunoglobulin binding polypeptide mimicking the binding activity of the CDR or parent binding domain. Insertion of three CDRs into distinct loops with spatial separation similar to an antibody variable region will produce a non-immunoglobulin binding polypeptide mimicking the binding activity of the parent variable region. Similarly, insertion of multiple binding domains that together contribute to binding of the parent polypeptide will yield a non-immunoglobulin binding polypeptide exhibiting binding activity of the multiple binding domains within the parent polypeptide. Therefore, non-immunoglobulin binding polypeptides of the invention can be produced where one, two, three, four, five, six or more, binding domains are inserted into a Ig-like domain containing scaffold to produce a chimeric non-immunoglobulin binding polypeptide of the invention.

When inserting multiple binding domains from a parent polypeptide into an Ig-like domain containing scaffold it is beneficial to conserve the spatial separation of the binding domains within the resulting chimeric non-immunoglobulin binding polypeptide. Because the parent and scaffold structures are conserved, spatial conservation will preserve the binding activity of the parent polypeptide in the resulting chimeric binding polypeptide. If desired, scaffold framework residues can be altered to, for example, augment or optimize the binding activity of a resultant chimeric non-immunoglobulin binding polypeptide. Such alterations are routine to those skilled in the art and constitute making one or more changes and testing the resulting molecule. The changes can be made sequentially or in parallel. The production of libraries of many different, including a hundred or hundreds, a thousand or thousands, or a million or millions, of variant members can be made and screened for the same or optimized binding activity in a very short period of time. Such methods are well known to those skilled in the art and can be used in conjunction with the methods described herein.

The production of non-immunoglobulin binding polypeptides having multiple inserted parent binding domains can be performed by selecting an Ig-like domain containing scaffold polypeptide and splicing the multiple binding domains into the loop or non-structural regions of a scaffold. Generating a non-immunoglobulin binding polypeptide that mimics the parent polypeptide can be performed by selecting a Ig-like domain containing scaffold that has at least the same number of loop regions as does the parent binding polypeptide. For the specific example of an antibody variable region parent binding polypeptide, a suitable Ig-like domain containing scaffold will have at least three loop regions. Each region to harbor one of the three CDRS. Production of a chimeric binding polypeptide of the invention mimicking the dimeric V_{H} and V_{L} Fᵥ fragment, a Ig-like domain containing scaffold can be selected to contain, for example, six loop regions. Alternatively, two different scaffolds can be produced with one scaffold containing CDRs corresponding to V_{H} CDRs 1-3 and the other scaffold containing CDRs corresponding th V_{L} CDRs 1-3. The two chimeric non-immunoglobulin binding polypeptides can then be brought together to form an antibody binding mimic having all six parent antibody CDRs by, for example, self-assembly where domains corresponding to binding partners are respectively included in each of the scaffold polypeptides. Alternatively, the two chimeric non-immunoglobulin binding polypeptides also can be brought together by inclusion of a linker sequence such as that used in single chain antibody formation.

Insertion of donor binding domains such as CDRs into a structurally analogous scaffold such as an Ig-like domain containing scaffold of the invention will result in a chimeric non-immunoglobulin binding polypeptide that mimics the binding activity of the parent antibody. In this regard, the chimeric non-immunoglobulin binding polypeptide will exhibit selective binding activity toward the ligand bound by the parent antibody. Accordingly, a chimeric non-immunoglobulin binding polypeptide constructed from antibody CDRs and an Ig-like domain containing scaffold will demonstrate, for example, sufficient binding specificity to discriminate the target ligand over non-target ligands.

Methods for insertion of CDRs can be performed at the level of the encoding nucleic acid or directly at the polypeptide level. In the former method, a nucleic acid encoding the desired polypeptide is generated and then the polypeptide is produced by, for example, in vitro translation or in vivo biosyntesis in a host cell or organism, both of which are well known in the art. In the latter method, the amino acid sequence corresponding to a chimeric non-immunoglobulin binding polypeptide of interest can be synthesized directly.

Method for constructing an encoding nucleic acid also are well known in the art. For example, encoding nucleic acids can be produced by any method of nucleic acid synthesis known to those skilled in the art. Such methods include, for example, chemical synthesis, recombinant synthesis, enzymatic polymerization and combinations thereof. These and other synthesis methods are well known to those skilled in the art.

For example, methods for synthesizing polynucleotides can be found described in, for example, Oligonucleotide Synthesis: A Practical Approach, Gate, ed., IRL Press, Oxford (1984); Weiler et al., Anal. Biochem. 243:218 (1996); Maskos et al., Nucleic Acids Res. 20:1679 (1992); Atkinson et al., Solid-Phase Synthesis of Oligodeoxyribonucleotides by the Phosphitetriester Method, in Oligonucleotide Synthesis 35 (M. J. Gait ed., 1984); Blackburn and Gait (eds.), Nucleic Acids in Chemistry and Biology, Second Edition, New York: Oxford University Press (1996), and in Ansubel et al., Current Protocols in Molecular Biology, John Wiley and Sons, Baltimore, Md. (1999).

Recombinant and enzymatic synthesis, including polymerase chain reaction and other amplification methodologies can be found described in, for example, Sambrook et al., Molecular Cloning: A Laboratory Manual, Third Ed., Cold Spring Harbor Laboratory, New York (2001) and in Ansubel et al., (1999), supra.

Solid-phase synthesis methods for generating numerous different polynucleotides and other polymer sequences can be found described in, for example, Pirrung et al., U.S. Pat. No. 5,143,854 (see also PCT Application No. WO 90/15070), Fodor et al., PCT Application No. WO 92/10092; Fodor et al., Science (1991) 251:767-777, and Winkler et al., U.S. Pat. No. 6,136,269; Southern et al. PCT Application No. WO 89/10977, and Blanchard PCT Application No. WO 98/41531. Such methods include synthesis and printing of arrays using micropins, photolithography and ink jet synthesis of polynucleotide arrays. Methods for efficient synthesis of nucleic acid polymers by sequential annealing of polynucleotides can be found described in, for example, in U.S. Pat. No. 6,521,437, to Evans.

Similarly, chemical synthesis of polypeptides also is well known to those skilled in the art. Accordingly, a chimeric non-immunoglobulin binding polypeptide of the invention can be synthesized directly using any of a variety of methods well known in the art. Such methods include, for example, those previously set forth and described in Roberts and Vellaccio (Academic Press, Inc.), supra; Wilson and Czarnik (John Wiley & Sons Inc.), supra; Manfred E. Wolff (John Wiley & Sons Inc.), supra; U.S. Pat. Nos. 5,420,109; 5,849,690; 5,686,567; 5,990,273; in PCT publication WO 01/00656, supra, and in M. Bodanzsky (Springer-Verlag), supra, and Stewart and Young (Pierce Chemical Co.), supra.

The invention also provides a chimeric non-immunoglobulin binding polypeptide having an immunoglobulin-like domain containing scaffold having less than about 20% sequence identity to a human immunoglobulin variable region framework domain, the immunoglobulin-like domain containing scaffold having two or more altered solvent exposed loops and exhibiting selective binding activity toward a disparate ligand.

As described previously, an Ig-like domain containing scaffold member selected from either the IgSF, fibronectin type III or caderin superfamily exhibits a sandwich structure having at least one loop region. Generally, an Ig-like domain containing scaffold will contain multiple loop regions including, for example, two, three, four, five or six or more loop regions that form a turn between β-sheets of the sandwich structure. Because the β-strands are packed by hydrophobic interactions the loop regions will generally be exposed to solvent and accessible for ligand binding. Accordingly, such solvent exposed loop regions within an Ig-like domain containing scaffold are amenable to transformation into a binding domain which confer binding activity onto the resultant scaffold polypeptide.

Because Ig-like domain containing scaffolds have been characterized according to their structure, rather than primary amino acid sequence, the degree of sequence similarity or identity is not determinative in generating a non-immunoglobulin binding polypeptide of the invention. However, a Ig-like domain containing scaffold of the invention is distinguishable in sequence identity from a human immunoglobulin framework sequence. Accordingly, an Ig-like domain containing scaffold of the invention can exhibit a wide range of sequence identity differences compared to a human immunoglobulin variable region framework. Such ranges include, for example, from about 60% identity to 20% or less sequence identity as well as all sequence identities between and below these numbers. In some instances, the sequence identity between an Ig-like domain containing scaffold and a human antibody framework will be higher than 60% and can include, for example, sequence identities of 65%, 70%, 75% or 80% or higher, so long as the scaffold amino acid sequence does not constitute a human antibody sequence. Exemplary Ig-like domain containing scaffolds have been described previously.

Transferring binding activity from a parent binding polypeptide to an Ig-like domain containing scaffold can be accomplished in a variety of ways. For example, and as described previously, the binding domains of a parent polypeptide can be inserted or transferred to the loop regions of an Ig-like domain containing scaffold. Although described previously with primary reference to CDRs of an antibody, such transferred binding domains can be derived from a variety of different parent binding polypeptides. Such parent binding polypeptides include, for example, those described above and below as well as other polypeptides well known in the art that exhibit a desired activity. Using the teachings and guidance provided herein, any of the binding domains contained within these polypeptides, or others well known to those skilled in the art, can be transferred to a Ig-like domain containing scaffold to produce a chimeric non-immunoglobulin binding polypeptide of the invention.

Alternatively, binding activity can be generated de novo within an Ig-like domain containing scaffold by altering one or more loop regions and then selecting a polypeptide that exhibits a desired binding activity. Alteration of amino acid sequence within a loop region can be performed, for example, to change some or all residues within one or more loop regions. The alterations can include, for example, from one to all twenty different naturally occurring amino acids at one or more positions. The alterations also can included, for example, incorporation of amino acid analogues and mimetics at any or all positions. The production of populations of molecules having altered amino acid sequences and subsequent identification of binding activity is well known to those skilled in the art. Accordingly, using the teachings and guidance provided herein a non-immunoglobulin binding polypeptide can be produced de novo by producing a library of altered solvent exposed loop region sequences within a Ig-like domain containing scaffold and then screening the library population for a desired binding activity.

Methods for making such changes in the encoding nucleic acid also are well known to those skilled in the art. Using such methods one can produce populations having essentially all possible combinations of codon sequences within one, two, three or four or more loop regions. Such methods include, for example, those described in U.S. Pat. Nos. 5,223,409, 5,403,484, which describe the synthesis of variegated codons, as well as in U.S. Pat. No. 6,521,437, to Evans, supra, which describes the direct synthesis and assembly of essentially any desired sequence or population of sequences. Other methods well known in the art for producing a large number of alterations in a known amino acid sequence or for generating a diverse population of variable or random altered sequences include, for example, degenerate or partially degenerate oligonucleotide synthesis. Codons specifying equal mixtures of all four nucleotide monomers, represented as NNN, results in degenerate synthesis. Whereas partially degenerate synthesis can be accomplished using, for example, the NNG/T codon. Such methods can be found described in, for example, Cwirla et al., Proc. Natl. Acad. Sci. USA, 87:6378-6382 (1990); Devlin et al., Science, 249:404-406 (1990), and Parmley and Smith, Gene, 73:305-318 (1988).

Binding activity can be generated in an Ig-like domain containing scaffold by, for example, altering amino acid residues in at least two solvent exposed loops. The number of residues to be altered within each loop region can range from one to many residues. For the de novo production of a chimeric non-immunoglobulin binding polypeptide mimicking an antibody variable region, the number of residues to be altered can be, for example, about the size of a CDR. Relative size according to spatial organization should be taken into account where, for example, the first CDR is generally smaller than the second CDR. Alternatively, where a binding mimic is not contemplated, the number of altered residues within each of the two or more loop regions can range from one to many, including all of the residues within one or more of the loop regions. The determination of the number of residues to alter can be made based on other considerations, such as desired size of the resultant population to screen or the amount of labor desired to commit to the screening and identification procedures.

Accordingly, generation of a chimeric non-immunoglobulin binding polypeptide can be accomplished by altering one, two, three, four, five, six, seven, eight, nine or ten or more residues, including 20, 30, 40 or 50 or more residues within a scaffold loop region. Various combinations of such residue alterations can occur, for example, in one, two or three or more solvent exposed loops, including four, five, or six or more loop regions of an Ig-like domain containing scaffold polypeptide. ThyOx family members are exemplary of Ig-like domain containing scaffold polypeptides having from one to many altered solvent exposed loops for the production of a non-immunoglobulin binding polypeptide. IgSF, fibronectin type III and cadehin superfamily members are exemplary of Ig-like domain containing scaffold polypeptides having from two to many altered solvent exposed loops for the production of a non-immunoglobulin binding polypeptide.

In addition, alterations of loop region sequences can be performed sequentially on individual molecules such as in a step-wise optimization procedure. Employing a sequential, rather than bulk or parallel approach, can be beneficial when there are a few molecules to be imparted with binding specificity or when there are a small number of residues to change. Alternatively, any of various combinations of the above approaches can be employed to produce a non-immunoglobulin binding polypeptide from the de novo alteration of a Ig-like domain containing scaffold polypeptide. Therefore, a chimeric non-immunoglobulin binding polypeptide of the invention can be produced from the screening of populations of scaffolds having altered loop regions, from the step-wise generation of a desired binding activity, or from application of both approaches.

The altered scaffold polypeptides generated as described above, for example, can be screened for the ability to bind a disparate ligand. Binding activity toward a disparate ligand can include binding activity selective for any ligand different from an activity exhibited in the parent Ig-like domain containing scaffold polypeptide. Once a molecule exhibiting selective binding toward a desired disparate ligand is identified the altered Ig-like domain containing scaffold polypeptide can be considered a non-immunoglobulin binding polypeptide of the invention.

Selective binding activity toward a disparate ligand can be essentially any activity of a polypeptide including, for example, binding activity, catalytic activity or structural activity. Other activities as well as specific activities within any of these categories are well known to those skilled in the art and can be considered an activity of a binding polypeptide toward a disparate ligand.

For the specific instance where a population of altered solvent exposed loops, once the populations of altered scaffold encoding nucleic acids have been constructed as described above, they can be expressed to generate a population of altered variable region polypeptides that can be screened for binding affinity. By analogy, the above and below described methods are equally applicable for the screening and identification of a desired binding activity for a scaffold having a single or a few alterations.

For example, scaffold encoding nucleic acids having altered loop regions can be cloned into an appropriate vector for propagation, manipulation and expression. Such vectors are known or can be constructed by those skilled in the art and should contain expression elements sufficient for the transcription, translation, regulation, and if desired, sorting and secretion of the altered scaffold polypeptides. The vectors also can be for use in either procaryotic or eukaryotic host systems so long as the expression and regulatory elements are of compatible origin. The expression vectors can additionally included regulatory elements for inducible or cell type-specific expression. One skilled in the art will know which host systems are compatible with a particular vector and which regulatory or functional elements are sufficient to achieve expression of the polypeptides in soluble, secreted or cell surface forms.

Appropriate host cells, include for example, bacteria and corresponding bacteriophage expression systems, yeast, avian, insect and mammalian cells. Methods for recombinant expression, screening and purification of populations of altered variable regions or altered variable region polypeptides within such populations in various host systems are well known in the art and are described, for example, in Sambrook et al., supra, and in Ansubel et al., supra. The choice of a particular vector and host system for expression and screening of altered variable regions will be known by those skilled in the art and will depend on the preference of the user.

The expressed population of Ig-like domain containing scaffolds having altered loop regions can be screened for the identification of one or more altered species exhibiting a predetermined binding affinity. Screening can be accomplished using various methods well known in the art for determining the binding affinity of a polypeptide or compound. Additionaly, methods based on determining the relative affinity of binding molecules to their partner by comparing the amount of binding between the altered scaffold polypeptides and the unaltered scaffold, for example, can similarly be used for the identification of a predetermined binding species. All of such methods can be performed, for example, in solution or in solid phase. Moreover, various formats of binding assays are well known in the art and include, for example, immobilization to filters such as nylon or nitrocellulose; two-dimensional arrays, enzyme linked immunosorbant assay (ELISA), radioimmune assay (RIA), panning and plasmon resonance. Such methods can be found described in, for example, Sambrook et al., supra, and Ansubel et al. Methods for measuring the affinity, including association and disassociation rates using surface plasmon resonance are well known in the art and can be found described in, for example, Jönsson and Malmquist, Advances in Biosnsors, 2:291-336 (1992) and Wu et al. Proc. Natl. Acad. Sci. USA, 95:6037-6042 (1998). Moreover, one apparatus well known in the art for measuring binding interactions is a BIAcore 2000 instrument which is commercially available through Pharmacia Biosensor, (Uppsala, Sweden).

Using any of the above described screening methods, as well as others well known in the art, an Ig-like domain containing scaffold having altered solvent exposed loop regions can be identified by detecting the binding of at least one altered variable region within the population to a predetermined antigen. Additionally, the above methods can alternatively be modified by, for example, the addition of substrate and reactants to identify altered variable regions having a predetermined catalytic activity. Those skilled in the art will know, or can determine, binding conditions which are sufficient to identify selective interactions over non-specific binding.

Detection methods for identification of binding species within the population of scaffolds having altered loop regions can be direct or indirect and can include, for example, the measurement of light emission, radioisotopes, calorimetric dyes and fluorochromes. Direct detection includes methods that operate without intermediates or secondary measuring procedures to assess the amount of bound antigen or ligand. Such methods generally employ ligands that are themselves labeled by, for example, radioactive, light emitting or fluorescent moieties. In contrast, indirect detection includes methods that operate through an intermediate or secondary measuring procedure. These methods generally employ molecules that specifically react with the antigen or ligand and can themselves be directly labeled or detected by a secondary reagent. For example, a non-immunoglobulin binding polypeptide specific for a ligand can be detected using a secondary antibody capable of interacting with the binding polypeptide specific for the ligand, again using the detection methods described above for direct detection. Indirect methods can additionally employ detection by enzymatic labels. Moreover, for the specific example of screening for catalytic non-immunoglobulin binding polypeptide, the disappearance of a substrate or the appearance of a product can be used as an indirect measure of binding affinity or catalytic activity.

The methods of synthesis, expression, screening and detection described above in reference to scaffold polypeptides having altered loop regions can be employed equally to the production and identification of non-immunoglobulin binding polypeptides having inserted CDRs or other binding domains. Such methods similarly are equally applicable to the production and identification of chimeric ThyOx binding polypeptides and of chimeric ThyOx carrier polypeptides described further below. Those skilled in the art will known, or can determine using the teachings and guidance provided herein, which methods can be used to construct or facilitate the construction of any of the various non-immunoglobulin binding polypeptides of the invention.

Therefore, the invention further provides a chimeric ThyOx binding polypeptide having one or more altered immunoglobulin-like domain loop regions of a ThyOx family polypeptide and having selective binding activity toward a non-ThyOx ligand. The chimeric ThyOx binding polypeptide can be derived from a ThyOx family scaffold polypeptide consisting of Ox2, CD7, Ox2-like protein or Ox2 homolog, or a functional fragment thereof. The ThyOx binding polypeptides can additionally be generated insertion of CDR binding domains or changing of amino acid sequence with the solvent exposed loop regions of its Ig-like domain containing scaffold structure.

The invention also provides a chimeric ThyOx carrier polypeptide having a at least one immunoglobulin-like domain containing scaffold derived from a ThyOx family polypeptide, and a heterologous binding polypeptide exhibiting selective binding activity toward a non-ThyOx ligand.

In addition to inserting one or more heterologous binding domains from a donor binding polypeptide or altering de novo one or more solvent loop regions of a Ig-like domain containing scaffold, a binding polypeptide also can be generated using a Ig-like domain containing polypeptide as a carrier polypeptide. Carrier polypeptides are useful, for example, as a chaperon vehicle to impart a variety of attributes onto a binding polypeptide. Such attributes include, for example, increasing stability, half-life, size or a combination of these or other attributes. Another attribute includes, for example, imparting a heterologous function onto a binding polypeptide. A ThyOx carrier binding polypeptide includes at least one Ig-like domain containing scaffold derived from a ThyOx family member polypeptide and a heterologous binding domain fused to the carrier portion of the polypeptide.

ThyOx family members are exemplary Ig-like domain containing polypeptides that can additionally be employed as a carrier polypeptide. As with other Ig-like domain containing polypeptides, ThyOx family members are stable under a variety of conditions because, for example, they contain one or more conserved Ig-like β-sandwich structures. Additionally, ThyOx family members also lack a known binding ligand. Accordingly, production of chimeric ThyOx carrier binding polypeptides are devoid of any known binding activity that can be imparted by the carrier portion of the ThyOx carrier polypeptide. Absence of a binding activity derived from the carrier portion of the chimeric polypeptide functions to increase the effective specificity of the chimeric ThyOx binding polypeptide.

Production of ThyOx carrier binding polypeptides can be performed by, for example, fusion of a ThyOx Ig-like domain containing scaffold to a heterologous binding domain. Any heterologous binding domain, or functional fragment thereof, that is capable of retaining binding activity when attached to a heterologous polypeptide such as a carrier polypeptide can be employed for the production of a chimeric ThyOx carrier binding polypeptide of the invention. Such binding domains will generally exhibit inherent structural integrity when segregated, for example, from its parent polypeptide. Alternatively, whole binding polypeptides can be fused to a ThyOx carrier polypeptide to yield a chimeric ThyOx carrier binding polypeptide of the invention. The choice of using whole binding polypeptides or functional fragments of a parent binding can depend, for example, on convenience as well as on the intended outcome. For example, where efficacy is sought to be optimized it is beneficial to reduce non-target binding specificity as much as possible. Specific examples of heterologous binding domains useful for producing a chimeric ThyOx carrier binding polypeptide of the invention include glucagon, glucagon-like peptide, erythropoietin, one or more antibody variable regions, or functional fragments thereof. Essentially, all other polypeptides exhibiting a desired activity residing in one or more domains or locations of a polypeptide can similarly be employed as a parent polypeptide for obtaining heterologous binding domains useful for producing chimeric ThyOx carrier binding polypeptides of the invention.

As with the non-immunoglobulin binding polypeptide described previously, fusion can occur at the encoding nucleic acid level, followed by synthesis or biosynthesis of the encoded polypeptide. Fusion also can occur by direct chemical synthesis of the polypeptide. Accordingly, all of the methods described previously are employed applicable for the production of a chimeric ThyOx carrier binding polypeptide of the invention.

The invention additionally provides a nucleic acid encoding a non-immunoglobulin, a ThyOx binding polypeptide, or a ThyOx carrier binding polypeptide of the invention. The translation of a nucleic acid into an amino acid sequence or the reverse translation of an amino acid into an encoding nucleic acid sequence is well known to those skilled in the art. Given either sequence it is a routine matter to translate or reverse translate between amino acid and nucleotide sequences because the genetic code has long been elucidated. Moreover, because the non-immunoglobulin binding polypeptide of the invention are chimeric many donor sequences will be known for both the binding domain portion as well as for the Ig-like domain containing scaffold portion.

For chimeric polypeptides constructed from a donor binding domains and scaffold, one skilled in the art will know either or both the amino acid or nucleotide sequence when designing the chimeric polypeptide. Therefore, the sequence of the final produce will also be known once the insertion boundaries or residues are determined. Specific examples of such simultaneous design and determination of the amino acid and nucleotide sequences are provided below in the Examples.

For chimeric polypeptides constructed by de novo alteration of one or more solvent exposed loop regions, the amino acid or encoding nucleotide sequence can be determined using methods well known in the art. In this regard, because the altered positions are predetermined due to the design process it is sufficient to determine the identity of residues at these altered positions to know the complete amino acid or nucleotide sequence. Unless specifically targeted for changes, the scaffold framework residues will be unaltered. Moreover, when starting from a scaffold of known sequence, resequencing the known residues is unnecessary. Therefore, it is sufficient to determine the sequence at the altered position of selected binders and substitute the newly identified residues into the known scaffold sequence.

Methods for sequencing an encoding non-immunoglobulin binding polypeptide nucleic acid is generally an efficient means to determine both the nucleotide and deduced, or translated, amino acid sequence. Such methods are well known in the art and available in fully automated formats. However, it can be desirable to first sequence the relevant polypeptide portion of the chimeric non-immunoglobulin binding polypeptide and then reverse-translate that sequence to obtain an encoding nucleotide sequence. The degeneracy of the genetic code can allow, for example, the inclusion of multiple different codons which encode the same amino acid. Any of such nucleotide sequence variants inherent in the genetic code are included in an encoding nucleic acid of the invention.

It is understood that modifications which do not substantially affect the activity of the various embodiments of this invention are also included within the definition of the invention provided herein. Accordingly, the following examples are intended to illustrate but not limit the present invention.

### EXAMPLE I

### Non-Immunoglobulin Binding Polypeptides Produced from Thy1 Scaffolds Harboring Antibody Binding Sequences

This example shows the production of a non-immunoglobulin binding polypeptide where antibody CDRs have been inserted into a Thy1 Ig-like domain containing scaffold.

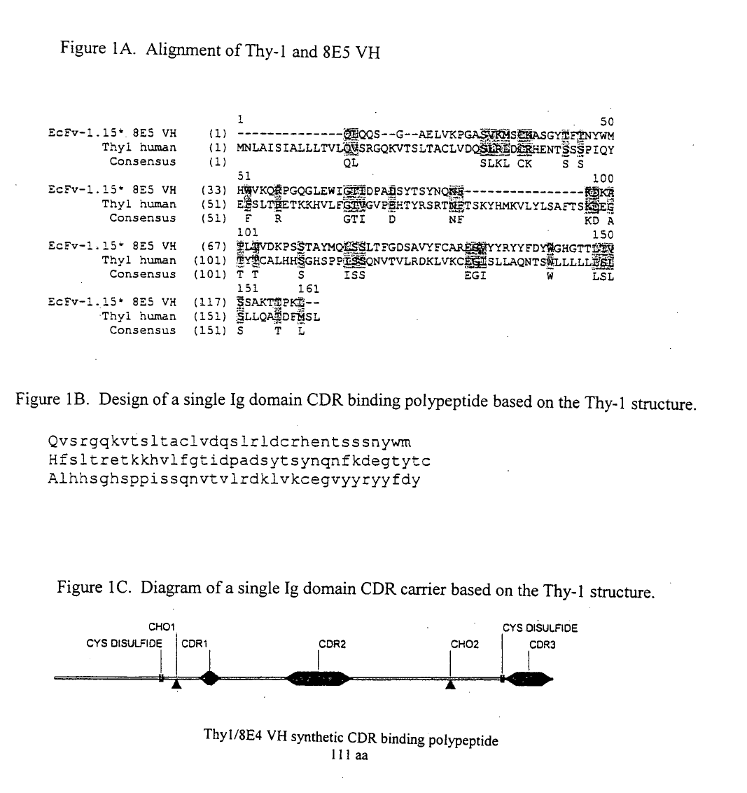

Production of a synthetic binding polypeptide for antibody CDRs was based on the Thy1 polypeptide which contains Ig-like folds. The human Thy-1 molecule was utilized as a scaffold for the insertion of antibody CDRs. Briefly, Thy1 is a molecule within the IgSF and contains 161 amino acids. An alignment of human Thy-1 with the V_{H} domain of the single chain antibody 8E5 is shown in FIG. 1A. Antibody 8E5 has anti-fibrin binding activity and is described in, for example, Song et al., Hybridoma 16:235-241 (1997). The locations of the CDR regions of 8E5 as well as other features relative to Thy1 is shown in the FIG. 1A. There is 16.8% identity between the two molecules (21 identities out of 125 residues) and 14.9% identity between the frameworks of 8E5 and the Thy1 scaffold (14 identical residues out of 100 framework residues).

The Thy1 derived non-immunoglobulin binding polypeptide was designed based on the amino acid sequence alignment to incorporate the following modifications. Briefly, the 16 amino acid loop located between amino acid residues 81 and 96 was removed. Residues 47-51 of Thyl were replaced with the CDR1 loop of 8E5 V_{H}, which correspond to V_{H} residues 29-34. Thy1 residues 67-98 were replaced with 8E5 V_{H} CDR2 residues 48-64. Thyl residues 130-140 similarly were replaced with 8E5 V_{H} CDR3 residues 98-109. The Thy1 leader peptide corresponding to amino acids 1-14 of the propeptide was removed. Finally, the transmembrane peptide corresponding to residues 141-161 of Thy1 was removed to ensure solubility. Alternatively, the transmembrane residues could be modified to make it less hydrophobic. The amino acid sequence of the resultant chimeric Thy1 non-immunoglobulin binding polypeptide is shown in FIG. 1B with a schematic of the resultant structure shown in FIG. 1C.

Synthesis of the above Thy1-containing CDR non-immunoglobulin binding polypeptide proceeded using automated synthesis and assembly of oligonucleotides encoding the chimeric non-immunoglobulin binding polypeptide having CDRs inserted into a Thy1 scaffold. Synthesis and assembly was performed as described, for example, in U.S. Pat. No. 6,521,427, and in PCT publications PCT/US98/1931 and PCT/US02/0164.

Briefly, a non-immunoglobulin binding polypeptide containing CDR sequences inserted into loop domains of the Ig-like domain containing scaffold Thy1 polypeptide was engineered as described above. Thy1 and 8E5 encoding nucleic acids were used as the genetic source of the nucleotide and amino acid sequences for the Thyl scaffold and the 8E5 donor CDRs.

The design and computer synthesis of this anti-fibrin Thyl non-immunoglobulin binding polypeptide was performed by electronically arranging the nucleotide sequences encoding the scaffold framework and CDR encoding regions into a single, contiguous nucleotide sequence encoding the resultant chimeric polypeptide described above. The resultant electronic version of the encoding nucleotide sequence for the anti-fibrin Thy1 binding polypeptide was then subjected to electronic parsing and chemically synthesized as described below.

Following computer synthesis as described above, the chimeric anti-fibrin Thy1 non-immunoglobulin binding polypeptide is chemically synthesized. Synthesis was accomplished by first electronically parsing the encoding chimeric sequence into smaller oligonucleotide sequences that can be more efficiently synthesized. The electronic parsing was performed for both the sense and complementary antisense strands of the chimeric anti-fibrin Thyl non-immunoglobulin binding polypeptide. Parsing also was performed by maintaining partial complementarity between the 5′ terminus of either the sense or antisense strand and the 31 terminus of its corresponding complementary sequence so that adjacent oligonucleotides could be annealed with a complementary oligonucleotide to form an overlapping oligonucleotide assembly for both strands that span the genome. The size of each parsed oligonucleotide can vary, but generally, will be between about 50-150 nucleotides (nt) in length with an about 50% overlap between complementary sense and antisense strands.

The above-described encoding sequence for the chimeric anti-fibrin Thy1 non-immunoglobulin binding polypeptide was parsed electronically using a computer algorithm and corresponding executable program which generates two sets of overlapping oligonucleotides. The oligonucleotides were parsed using Parseoligo™, a proprietary computer program that optimizes nucleic acid sequence assembly. Optional steps in sequence assembly can include identifying and eliminating sequences that can give rise to hairpins, repeats or other difficult sequences. Additionally, the algorithm can first direct the synthesis of coding regions to correspond to a desired codon preference. For conversion of the chimeric anti-fibrin Thy1 polypeptide sequence to another codon preference, the algorithm utilizes a polypeptide sequence to generate a DNA sequence using a specified codon table. The algorithm for this step is can be described as follows:

For the DNA sequence GENE[ ], an array of bases, is generated from the protein sequence AA [ ], an array of amino acids, using a specified codon table.

a. parameters

i. N Length of protein in amino acid residues

ii. L=3N Length of gene in DNA bases

iii. Q Length of each component oligonucleotide

iv. X=Q/2 Length of overlap between oligonucleotides

v. W=3N/Q Number of oligonucleotides in the F set

vi. Z=3N/Q+1 Number of oligonucleotides in the R set

vii. F [1:W] set of (+) strand oligonucleotides

viii. R[L:Z] set of (−) strand oligonucleotides

ix. AA [1:N] array of amino acid residues

x. GENE [1:L] array of bases comprising the gene

b. Obtain or design a protein sequence AA [ ] consisting of a list of amino acid residues.

c. Generate the DNA sequence, GENE [ ], from the protein sequence, AA [ ]

i. For I=1 to N

ii. Translate AA [J] from codon table generating GENE[I: I+2]

iii. I=I+3

iv. J=J+1

V. Go to ii

With or without specifying a codon preference for coding regions, the parsing algorithm generated a set of parsed oligonucleotides corresponding to the entire length of the sense and antisense stand of the chimeric anti-fibrin Thy1 polypeptide encoding gene. The parsing was performed on the entire encoding sequence and optionally on flanking untranslated region sequences or vector sequences as needed. When polymerase chain reaction (PCR) is employed in the assembly process, for example, the parsing can be performed on about 10-15 kb fragments of the genome because this size is within the extension range of polymerases used in the procedure. However, PCR assembly augmentation was not needed in this procedure due to the small size of the non-immunoglobulin binding polypeptide. Parsing of the chimeric Thyl encoding nucleotide sequence resulted in about six different sets of sense and antisense oligonucleotides. These sets were assembled following the method described below, but without using PCR, described below and then ligated together to yield the completed basic genetic operating system. The parsing algorithm can be described as follows:

Two sets of overlapping oligonucleotides are generated from GENE [ ]; F [ ] covers the sense strand and R [ ] is a complementary, partially overlapping set covering the antisense strand.

a. Generate the F[ ] set of oligos

i. For I=1 to W

ii. F [I]=GENE [I:I+Q−1]

iii. I═I+Q

iv. Go to ii

b. Generate the R set of oligos

i. J=W

ii. For I=1 to W

iii. R[I]=GENE [W:W−Q]

iv. J=J−Q

v. Go to iii

c. Result is two set of oligos F [ ] and R [ ] of Q length

d. Generate the final two finishing oligos

i. S[l]=GENE [Q/2:1]

ii. S[2]=GENE [L−Q/2:L]

Following electronic parsing, automated synthesis of the individual oligonucleotides using phosphoramidite oligonucleotide synthesis chemistry was then performed. Automated assembly of the oligonucleotides into the chimeric anti-fibrin Thy1 non-immunoglobulin binding polypeptide was accomplished by sequentially annealing and ligating partially complementary oligonucleotides to result in the complete physical synthesis of the encoding nucleic acid of about 333 base pairs (bp) in length. All of the above steps are described in further detail below.

Briefly, the computer output of the parsed set of oligonucleotides for both the sense and antisense strand of the chimeric anti-fibrin Thy1 encoding nucleic acid was transferred to oligonucleotide synthesizer driver software. The synthesis of sequences of about 100 nt in length was manufactured and assembled using an array synthesizer system and used without further purification. For example, two 96-well plates containing 100 nt oligonucleotides can yield a 9600 bp fragment of a gene cassette. Therefore, synthesis of large nucleic acids can be performed using a singly 96-well plate. Once synthesized, the individual oligonucleotides can be maintained in the original plates or transferred to new multi-well format plates for oligonucleotide assembly.

Assembly was accomplished using, for example, robotics or microfluidics well known in the art for manipulating large numbers of oligonucleotide samples. Robotics and microfluidics allow synthesis and assembly to be performed rapidly and in a highly controlled manner. Such methods are described, for example, in WO 99/14318 and in U.S. Application Ser. Nos. 60/262,693 and 09/922,221.

For example, oligonucleotide parsing from the gene sequence designed in the computer were programmed for synthesis where sense and antistrands were placed in alternating wells of an array. Following synthesis in this format, the 12 rows of sequences of the gene are directed into a pooling manifold that systematically pools three wells into reaction vessels forming an annealed triplex structure. Following temperature cycling for annealing and ligation, four sets of annealed triplex oligonucleotides were pooled into 2 sets of 6 oligonucleotide products, then 1 set of 12 oligonucleotide products, depending on the size of the gene synthesized. Each row of the synthetic array was associated with a similar manifold resulting in the first stage of assembly of 8 sets of assembled oligonucleotides representing 12 oligonucleotides each. The second manifold pooling stage is controlled by a single manifold that pools the 8 row assemblies into a single complete assembly. Passage of the oligonucleotide components through the two manifold assemblies (the first 8 and the second single) results in the complete assembly of all 96 oligonucleotides from the array. The assembly module of Genewriter™ can include a complete set of 7 pooling manifolds produced using microfabrication in a single plastic block that sits below the synthesis vessels. Various configurations of the pooling manifold will allow assembly of 96, 384 or 1536 well arrays of parsed component oligonucleotides. A similar strategy can be performed where pairs of oligonucleotides are pooled instead of triplets.

An algorithm which can be implemented in a computer program for assembly of oligonucleotides as described above can be described as follows:

Two sets of oligonucleotides F[1:W] R[1:Z] S[1:2]

Step 1

a. For I=1 to W

b. Anneal F[I], F[I+1], R[I]; place in T[I]

c. Anneal F[I+2], R[I+1], R[I+2] T[I+1]

d. I=I+3

e. Go to b

Step 2

a. Do the following until only a single reaction remains

i. For I=1 to W/3

ii. Ligate T[I], T[I+1]

iii. I=I+2

iv. Go to ii

Described further below is the assembly of parsed oligonucleotides corresponding to the chimeric anti-fibrin Thy1 polypeptide described above following array synthesis of the oligonucleotide sets using a multi-well format. The method additionally employs polymerase chain reaction (PCR) in a two-step procedure to facilitate assembly.

Arrayed sets of parsed overlapping oligonucleotides are obtained by robotic instruments. Each oligonucleotide consisted of 50 nts with an overlap of about 25 base pairs (bp). The oligonucleotide concentration was about 250 nM (250 μM/ml). Fifty base oligos give Tms from 75 to 85 degrees C., 6 to 10 OD260, 11 to 15 nanomoles, 150 to 300 μg. Resuspend in 50 to 100 μl of H2O to make 250 nM/ml. Equal amounts of each oligonucleotide were combined to a final concentration of 250 μM (250 nM/ml) by adding 1 μl of each to give 192 μl. Addition of 8 μl dH2O followed to bring the volume up to 200 μl and a final concentration of 250 μM mixed oligos. The mixture was diluted 250-fold by taking 10 μl of mixed oligos and adding to 1 ml of water (1/100; 2.5 mM) followed by transferring 1 μl of this mixture into 24 μl 1× PCR mix. The PCR reaction includes: 10 mM TRIS-HCl, pH 9.0; 2.2 mM MgCl2; 50 mM KCl; 0.2 mM each dNTP, and 0.1 Triton X-100. One U TaqI polymerase is added to the reaction. The reaction is thermoycled under the following conditions for assembly: 55 cycles of (1) 94 degrees 30 s; (2) 52 degrees 30 s, and (3) 72 degrees 30 s.

Following assembly amplification, 2.5 μl of the assembly mix is added to 100 μl of PCR mix (40× dilution). Outside primers are prepared by taking 1 μl of F1 (forward primer) and 1 μl of R96 (reverse primer) at 250 μM (250 nm/ml-0.250 nmole/μl) and adding to the 100 μl PCR reaction. This mixture provides a final concentration of 2.5 μM each oligo. Taq1 polymerase is added (1U) and the reaction is thermocycle under the following conditions: 35 cycles (or original protocol 23 cycles) for (1) 94 degrees for 30 s; (2) 50 degrees for 30 s, and (3) 72 degrees for 60 s. The product is extract with phenol/chloroform, precipitate with ethanol and the pellet is resuspended in 10 μl of dH2O and analyze on an agarose gel.

An alternative method for assembly of parsed oligonucleotides corresponding to the chimeric anti-fibrin Thyl polypeptide described above following array synthesis of oligonucleotide sets is provided below. The method assembles parsed oligonucleotides using a Taq1 ligation procedure.

Briefly, arrayed sets of parsed overlapping oligonucleotides of about 25 to 150 bases in length each, with an overlap of about 12 to 75 base pairs (bp), are obtained. The oligonucleotide concentration is from 250 nM (250 μM/ml). For example, 50 base oligos give Tms from 75 to 85 degrees C., 6 to 10 od260, 11 to 15 nanomoles, 150 to 300 μg. The oligonucleotides are resuspended in 50 to 100 ml of H2O to make 250 nM/ml.

Using a robotic workstation, for example, a Beckman Biomek automated pipetting robot, or another automated lab workstation, equal amounts of forward and reverse oligonucleotides are combined pairwise. Equal volumes (10 μl) of forward and reverse oligonucleotides are mixed in a new 96-well v-bottom plate to provide one array with sets of duplex oligonucleotides at 250 μM, according to pooling scheme Step 1 in Table 2. An assembly plate is prepared by taking 2 μl of each oligomer pair and adding to a fresh plate containing 100 μl of ligation mix in each well. This procedure gives an effective concentration of 2.5 μM or 2.5 nM/ml. From each well of these wells, 20 μl is transferred to a fresh microwell plate and 1 μl of T4 polynucleotide kinase and 1 μl of 1 mM ATP subsequently added to each well. Each reaction will have 50 pmoles of oligonucleotide and 1 nmole ATP. The reactions are incubated at 37 degrees C. for 30 minutes.

Initiation of assembly is performed according to Steps 2-7 of Table 2. For example, pooling Step 2 is performed by mixing each successive well with the next. Taq1 ligase (1 μl) is then added to each mixed well and the mixture is cycled once at 94 degrees for 30 sec; 52 degrees for 30 s; then 72 degrees for 10 minutes.

Further assembly is performed according to step 3 of Table 2 of the pooling scheme and cycle according to the temperature scheme described above. Similarly, steps 4 and 5 of the pooling scheme are subsequently performed for further assembly and also cycled according to the temperature scheme above. Subsequent performance of step 6 of the pooling scheme is accomplished by transferring 10 μl of each mix into a fresh microwell and step 7 of the pooling scheme is accomplished by pooling the remaining three wells. The reaction volumes for each of these step within the pooling scheme will be:

Initial plate has 20 μl per well.

| | |
|---|---|
| Step 2 | 20 ul + 20 ul = 40 ul |
| Step 3 | 80 ul |
| Step 4 | 160 ul |
| Step 5 | 230 ul |
| Step 6 | 10 ul + 10 ul = 20 ul |
| Step 7 | 20 + 20 + 20 = 60 ul final reaction volume |

A final PCR amplification is then performed by taking 2 ul of final ligation mix and add to 20 ul of PCR mix containing 10 mM TRIS-HCl, pH 9.0, 2.2 mM MgCl2, 50 mM KCl, 0.2 mM each dNTP and 0.1% Triton X-100.

The outside primers are prepared by taking 1 μl of F1 (forward primer) and 1 μl of R96 (reverse primer) at 250 μM (250 nm/ml-0.250 nmole/μl) and add to the 100 μl PCR reaction giving a final concentration of 2.5 uM each oligo. Add 1 U Taq1 polymerase and cycle for 35 cycles under the following conditions: 94 degrees for 30 s; 50 degrees for 30 s; and 72 degrees for 60 s. The mixture is extracted with phenol/chloroform and precipitated with ethanol. The pellet is resuspend in 10 μl of dH2O and analyze on an agarose gel.

TABLE 2
Pooling scheme for ligation assembly.
Ligation method - Well pooling scheme

| STEP | FROM | TO |
|---|---|---|
| 1 | All F | All R |
| 2 | A1 | A2 |
| | A3 | A4 |
| | A5 | A6 |
| | A7 | A8 |
| | A9 | A10 |
| | A11 | A12 |
| | B1 | B2 |
| | B3 | B4 |
| | B5 | B6 |
| | B7 | B8 |
| | B9 | B10 |
| | B11 | B12 |
| | C1 | C2 |
| | C3 | C4 |
| | C5 | C6 |
| | C7 | C8 |
| | C9 | C10 |
| | C11 | C12 |
| | D1 | D2 |
| | D3 | D4 |
| | D5 | D6 |
| | D7 | D8 |
| | D9 | D10 |
| | D11 | D12 |
| | E1 | E2 |
| | E3 | E4 |
| | E5 | E6 |
| | E7 | E8 |
| | E9 | E10 |
| | E11 | E12 |
| | F1 | F2 |
| | F3 | F4 |
| | F5 | F6 |
| | F7 | F8 |
| | F9 | F10 |
| | F11 | F12 |
| | G1 | G2 |
| | G3 | G4 |
| | G5 | G6 |
| | G7 | G8 |
| | G9 | G10 |
| | G11 | G12 |
| | H1 | H2 |
| | H3 | H4 |
| | H5 | H6 |
| | H7 | H8 |
| | H9 | H10 |
| | H11 | H12 |
| 3 | A2 | A4 |
| | A6 | A8 |
| | A10 | A12 |
| | B2 | B4 |
| | B6 | B8 |
| | B10 | B12 |
| | C2 | C4 |
| | C6 | C8 |
| | C10 | C12 |
| | D2 | D4 |
| | D6 | D8 |
| | D10 | D12 |
| | E2 | E4 |
| | E6 | E8 |
| | E10 | E12 |
| | F2 | F4 |
| | F6 | F8 |
| | F10 | F12 |
| | G2 | G4 |
| | G6 | G8 |
| | G10 | G12 |
| | H2 | H4 |
| | H6 | H8 |
| | H10 | H12 |
| 4 | A4 | A8 |
| | A12 | B4 |
| | B8 | B12 |
| | C4 | C8 |
| | C12 | D4 |
| | D8 | D12 |
| | E4 | E8 |
| | E12 | F4 |
| | F8 | F12 |
| | G4 | G8 |
| | G12 | H4 |
| | H8 | H12 |
| 5 | A8 | B4 |
| | B12 | C8 |
| | D4 | D12 |
| | E8 | F4 |
| | F12 | G8 |
| | H4 | H12 |
| 6 | B4 | C8 |
| | D12 | F4 |
| | G8 | H12 |
| 7 | C8 | F4 |

Another alternative method for assembly of parsed oligonucleotides corresponding to the chimeric anti-fibrin Thy1 polypeptide described above following array synthesis of oligonucleotide sets is additionally described below. This method assembles parsed oligonucleotides using a TaqI synthesis and stepwise assembly.

Briefly, arrayed sets of parsed overlapping oligonucleotides of about 25 to 150 bases in length each, with an overlap of about 12 to 75 base pairs (bp), are obtained as described above and resuspended in 50 to 100 ml of H2O to make 250 nM/ml. Similarly, manipulations of samples is performed using robotics as described previously.

Two working multi-well plates containing forward and reverse oligonucleotides in a PCR mix at 2.5 mM are prepared and 1 μl of each oligo are added to 100 μl of PCR mix in a fresh microwell providing one plate of forward and one of reverse oligos in an array. Cycling assembly is then initiated as follows according to the pooling scheme outlined in Table 3. In the present example, 96 cycles of assembly can be accomplished according to this scheme.

To begin assembly, 2 μl of oligonucleotides in well F-E1 is transferred to a fresh well. Similarly, 2 μl of oligonucleotides in well R-E1 is transferred to a fresh well and 18 μl of 1×PCR mix and 1 U of Taq1 polymerase are added. The mixture is cycled once under the following conditions: (1) 94 degrees for 30 s; (2) 52 degrees for 30 s, and (3) 72 degrees for 30 s. Subsequently, 2 μl of oligonucleotides from well F-E2 and from well R-D12 is transferred to the reaction vessel. The mixture is cycled once according to the temperatures conditions described above. The pooling and cycling is repeated according to the scheme outlined in Table 3 for about 96 cycles.

A PCR amplification is then performed by taking 2 μl of final reaction mix and adding it to 20 μl of a PCR mix comprising: 10 mM TRIS-HCl, pH 9.0; 2.2 mM MgCl2; 50 mM KCl; 0.2 mM each dNTP, and 0.1° Triton X-100.

Outside primers are prepared by taking 1 μl of Fl and 1 ml of R96 at 250 mM (250 nm/ml-0.250 nmole/ml) and adding to the above 100 μl PCR reaction. This procedure yields a final concentration of 2.5 μM each oligonucleotide. 1 U Taq1 polymerase is subsequently added and the reaction is cycled for about 23 to 35 cycles under the following conditions: (1) 94 degrees for 30 s; (2) 50 degrees for 30 s, and (3) 72 degrees for 60 s. The reaction is subsequently extracted with phenol/chloroform, precipitated with ethanol and resuspend in 10 ml of dH2O for analysis on an agarose gel.

For initial pooling of the oligonucleotides, equal amounts of forward and reverse oligonucleotide pairs are added by taking 10 μl of forward and 10 μl of reverse oligonucleotide and mixing in a new 96-well v-bottom plate. This procedure provides one array with sets of duplex oligonucleotides at 250 mM, according to pooling scheme Step 1 in Table 3. An assembly plate is prepared by taking 2 μl of each oligomer pair and adding them to the plate containing 100 μl of ligation mix in each well. This gives an effective concentration of 2.5 μM or 2.5 nM/ml. About 20 μl of each well is transferred to a fresh microwell plate in addition to 1 μl of T4 polynucleotide kinase and 1 μl of 1 mM ATP. Each reaction will have 50 pmoles of oligonucleotide and 1 nmole ATP. The reaction is incubated at 37 degrees for 30 minutes.

Nucleic acid assembly was initiated according to Steps 2-7 of Table 3. For step 2, pooling is carried out by mixing each well with the next well in succession. Specifically, 1 μl of Taq1 ligase to is added to each mixed well and cycled once as follows: (1) 94 degrees for 30 sec; (2) 52 degrees for 30 s, and (3) 72 degrees 10 minutes.

Subsequently, step 3 of pooling scheme is carried out and cycled according to the temperature scheme described above. In like manner, steps 4 and 5 of the pooling scheme are then carried out and cycled according to the temperature scheme above. Step 6 of the pooling scheme is performed by taking 10 μl of each mix into a fresh microwell. Pooling the remaining three wells completes performance of step 7 of the pooling scheme. The reaction volumes will be (initial plate has 20 μl per well):

| | |
|---|---|
| Step 2 | 20 μl + 20 μl = 40 μl |
| Step 3 | 80 μl |
| Step 4 | 160 μl |
| Step 5 | 230 μl |
| Step 6 | 10 μl + 10 μl = 20 μml |
| Step 7 | 20 + 20 + 20 = 60 μl final reaction volume |

Following completion of the steps described above, a final PCR amplification is performed by taking 2 μl of the final ligation mix and adding it to 20 μl of PCR mix comprising: 10 mM TRIS-HCl, pH 9.0; 2.2 mM MgCl2; 50 mM KCl; 0.2 mM each dNTP, and 0.1% Triton X-100.

Outside primers are prepared by taking 1 μl of Fl and 1 μl of R96 at 250 mM (250 nm/ml-0.250 nmole/ml) and adding them to the above PCR reaction above giving a final concentration of 2.5 uM for each oligonucleotide. Subsequentlly, 1 U of Taq1 polymerase is added and cycled for about 23 to 35 cycles under the following conditions: (1) 94 degrees for 30 s; (2) 50 degrees for 30 s, and (3) 72 degrees for 60 s. The product is extracted with phenol/chloroform, precipitate with ethanol, resuspend in 10 μl of dH2O and analyzed on an agarose gel.

TABLE 3
Pooling scheme for assembly using
Taq1 polymerase (also topoisomerase II).

| | | |
|---|---|---|
| Step | Forward oligo | Reverse oligo |

| | | | | | |
|---|---|---|---|---|---|
| 1 | F | E | 1 + R | E | 1 Pause |
| 2 | F | E | 2 + R | D | 12 Pause |
| 3 | F | E | 3 + R | D | 11 Pause |
| 4 | F | E | 4 + R | D | 10 Pause |
| 5 | F | E | 5 + R | D | 9 Pause |
| 6 | F | E | 6 + R | D | 8 Pause |
| 7 | F | E | 7 + R | D | 7 Pause |
| 8 | F | E | 8 + R | D | 6 Pause |
| 9 | F | E | 9 + R | D | 5 Pause |
| 10 | F | E | 10 + R | D | 4 Pause |
| 11 | F | E | 11 + R | D | 3 Pause |
| 12 | F | E | 12 + R | D | 2 Pause |
| 13 | F | F | 1 + R | D | 1 Pause |
| 14 | F | F | 2 + R | C | 12 Pause |
| 15 | F | F | 3 + R | C | 11 Pause |
| 16 | F | F | 4 + R | C | 10 Pause |
| 17 | F | F | 5 + R | C | 9 Pause |
| 18 | F | F | 6 + R | C | 8 Pause |
| 19 | F | F | 7 + R | C | 7 Pause |
| 20 | F | F | 8 + R | C | 6 Pause |
| 21 | F | F | 9 + R | C | 5 Pause |
| 22 | F | F | 10 + R | C | 4 Pause |
| 23 | F | F | 11 + R | C | 3 Pause |
| 24 | F | F | 12 + R | C | 2 Pause |
| 25 | F | G | 1 + R | C | 1 Pause |
| 26 | F | G | 2 + R | B | 12 Pause |
| 27 | F | G | 3 + R | B | 11 Pause |
| 28 | F | G | 4 + R | B | 10 Pause |
| 29 | F | G | 5 + R | B | 9 Pause |
| 30 | F | G | 6 + R | B | 8 Pause |
| 31 | F | G | 7 + R | B | 7 Pause |
| 32 | F | G | 8 + R | B | 6 Pause |
| 33 | F | G | 9 + R | B | 5 Pause |
| 34 | F | G | 10 + R | B | 4 Pause |
| 35 | F | G | 11 + R | B | 3 Pause |
| 36 | F | G | 12 + R | B | 2 Pause |
| 37 | F | H | 1 + R | B | 1 Pause |
| 38 | F | H | 2 + R | A | 12 Pause |
| 39 | F | H | 3 + R | A | 11 Pause |
| 40 | F | H | 4 + R | A | 10 Pause |
| 41 | F | H | 5 + R | A | 9 Pause |
| 42 | F | H | 6 + R | A | 8 Pause |
| 43 | F | H | 7 + R | A | 7 Pause |
| 44 | F | H | 8 + R | A | 6 Pause |
| 45 | F | H | 9 + R | A | 5 Pause |
| 46 | F | H | 10 + R | A | 4 Pause |
| 47 | F | H | 11 + R | A | 3 Pause |
| 48 | F | H | 12 + R | A | 2 Pause |

The above-described Thy1/8E5 non-immunoglobulin binding polypeptide is one example showing the production of a chimeric binging polypeptide for one set of three CDRs from one immunlglobulin V_{H} or V_{L} based on a single Ig-like domain containing structure, Thyl. Other single domain structures as in Table 1 also can be used. Similarly, a two domain structure such as OX-2 can be used to derive a non-immunoglobulin binding polypeptide for two sets of 3 CDRs (CDRV_{L} and CDRV_{H}) making a two domain non-immunoglobulin binding polypeptide. This scaffold is similar to the form of scFv single chain synthetic antibodies. Other two domain structures in addition to OX-2 can be used for the basis of these designs.

### EXAMPLE II

### Non-Immunoglobulin Erythropoietin-Thy1 Carrier Binding Polypeptides

This Example shows the design and synthesis of a non-immunoglobulin Epo-Thyl carrier binding polypeptide for expression in CHO or other mammalian cells.

Epo (erythropoietin) is an important therapeutic which stimulates the production of red blood cells. There is a desire in the therapeutic industry to produce second generation drugs with longer serum half-lives and minimal immunoreactivity. Aranesp is a second generation Epo with five amino acid substitutions adding two additional glycosylation sites. The serum half-life of native Epo (Epogen) is three hours and the half-life of Aranesp is seven hours in rats. Fusing the Epo sequence with another highly glycosylated carrier is described below as a way to extend half-life without affecting innate activity.

Thy1 is a soluble, highly glycosylated T cell and neural polypeptide as a carrier for Epo. Advantages are that it is non-immunogenic and well tolerated by humans, exhibits long serum half-life and consists of a single chain carrier.

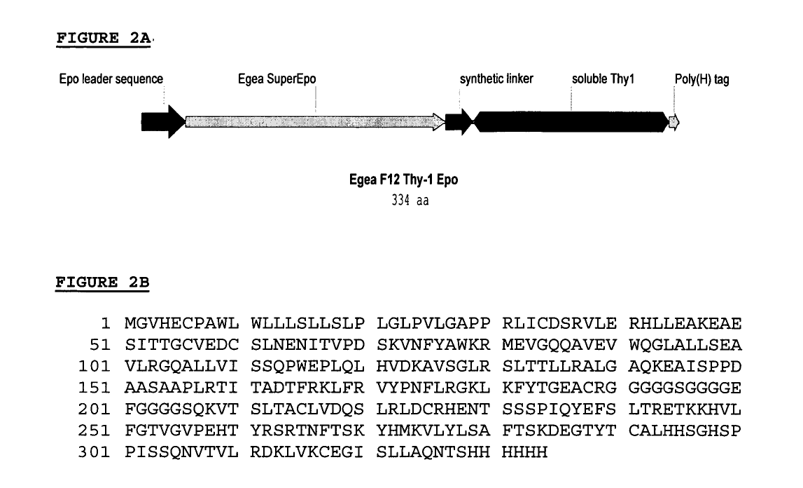

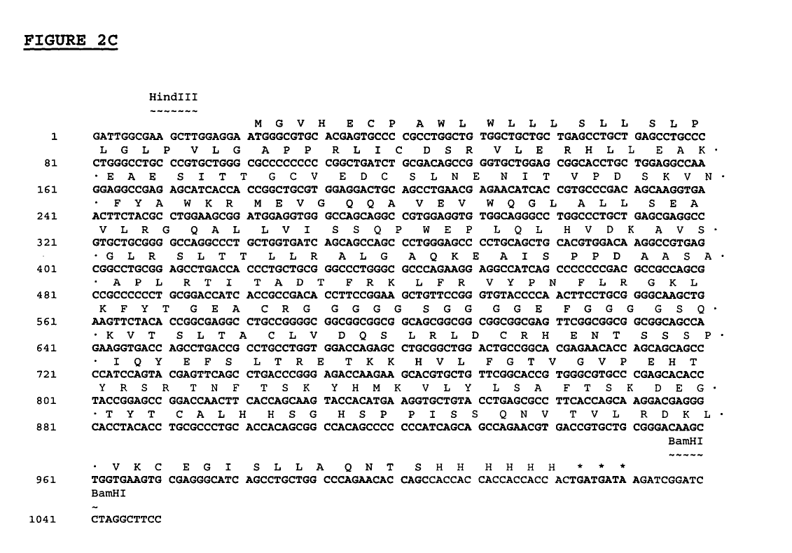

A non-immunoglobulin Epo-Thyl carrier binding polypeptide was designed to consist of the following components: modified human Epo at the amino terminal end of the mature polypeptide consisting of amino acid substitutions increase activity (“superEpo”); a human Epo leader sequence at the amino terminus, followed by a synthetic linker (GGGGS)₃; followed by mature human soluble Thy-1 (without Thy-1 leader sequence and transmembrane tail), and the inclusion of a 6× His tag at the carboxyl terminal end of the molecule. A schematic diagram of the resultant chimeric Epo-Thyl carrier binding polypeptide is shown in FIG. 2A.

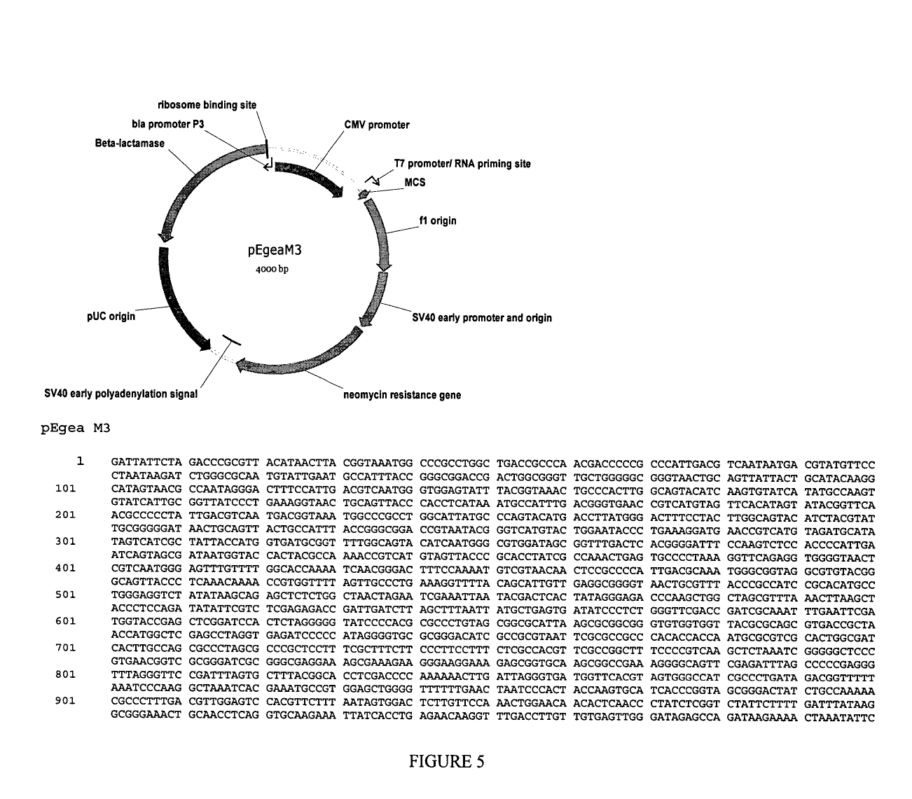

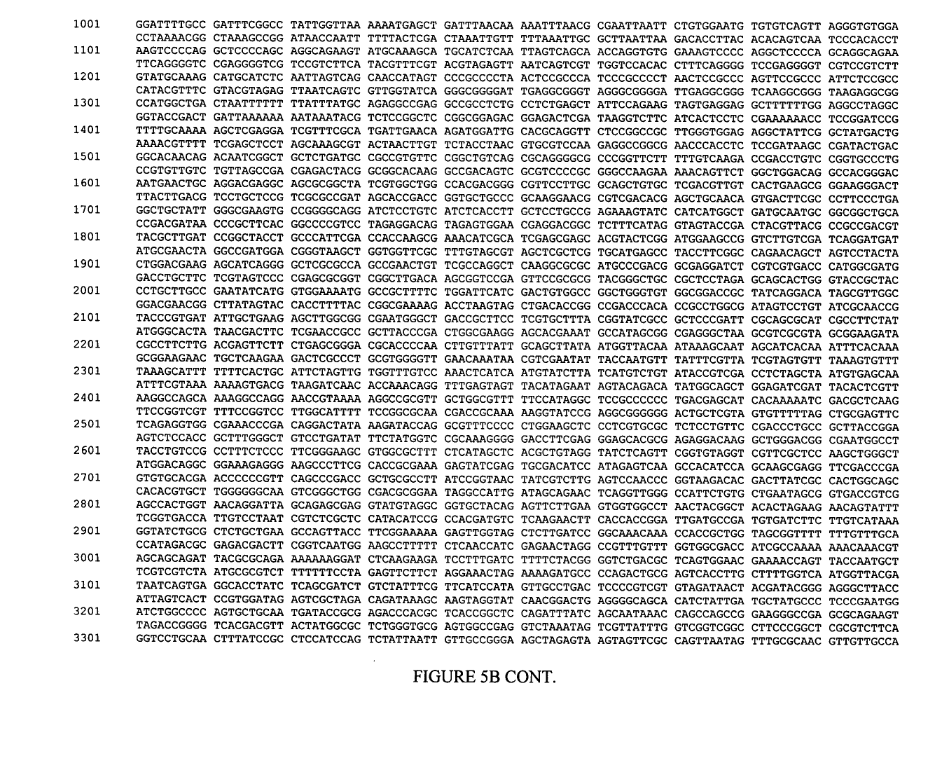

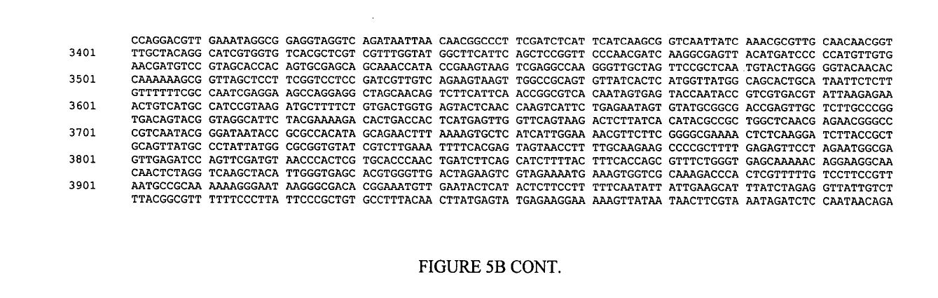

The encoding nucleic acid for the above design consists of 1050 bp. It was synthesized and incorporated into an expression vector, termed pEgeaM3, which is a mammalian expression vector using a CMV modified promoter for expression in CHO cells or other mammalian cells. A schematic diagram of pEgeaM3 and its nucleotide sequence is shown in FIG. 5. The amino acid sequence of the chimeric Epo-Thyl carrier binding polypeptide is shown in FIG. 2B. The encoding nucleotide sequence and corresponding amino acid sequence of the Epo-Thyl carrier binding polypeptide is shown in FIG. 2C.

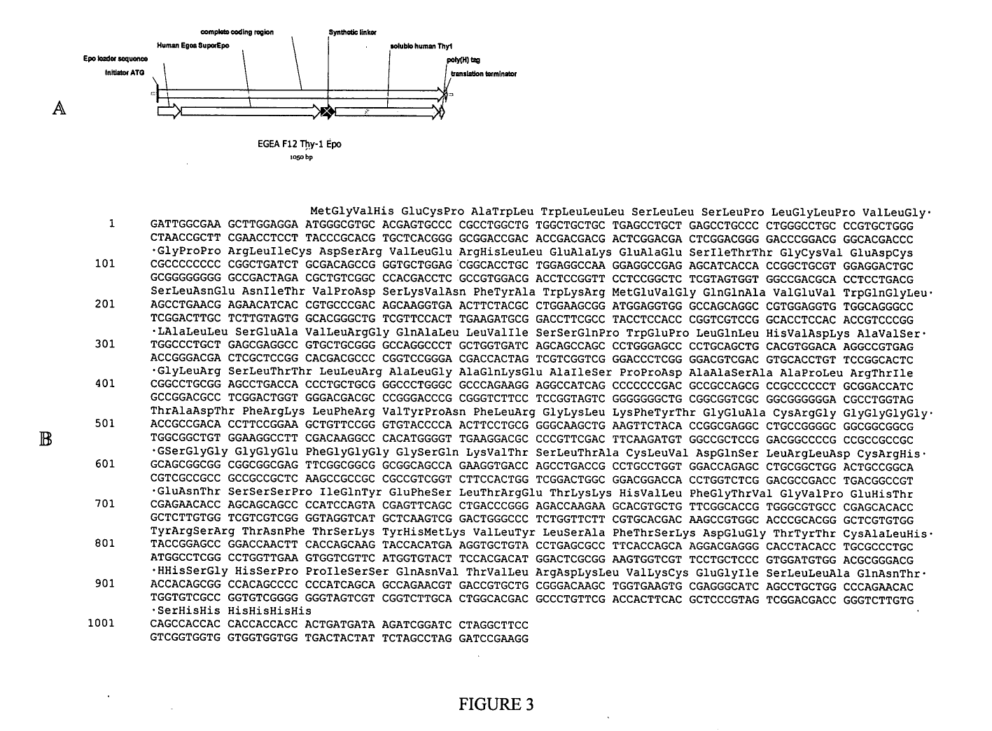

A Human SuperEpo construct was used as a starting sequence. The nucleotide and amino acid sequence of SuperEpo in context of the Epo-Thyl carrier binding polypeptide is shown in FIG. 3. FIG. 3 also provides a schematic diagram of this carrier binding polypeptide for reference to the sequence.

For construction of the non-immunoglobulin Epo-Thyl carrier binding polypeptide the human Epo leader was retained for expression in mammalian cells. A synthetic linker was incorporated following the leader that was based on the anti-fibrin single chain antibody (GGGGS)₃ synthetic linker in ScFv1.9.

The Thy1 sequence used was taken from the NCHI database which corresponds to the human preprotein version. The leader sequence and transmembrane regions were removed. All three carbohydrate (CHO) addition sites were retained, as was the entire Ig-like domain of the scaffold polypeptide, which can be modeled based on a single antibody variable region.

To produce the encoding nucleotide sequence, gene back-translation was performed using GeneWriter Gold 0.7 and Homo sapiens codon preference as described previously in Example I. Further, a 6× poly His (H) was added at the carboxy terminal to aid in protein purification. Three termination codons were also added to the final encoding nucleic acid. Finally, because no internal Bam Hl or Hind III site were present in the DNA sequence, these restriction sites can serve as convenient sites for cloning or excision of the non-immunoglobulin Epo-Thyl carrier binding polypeptide. Accordingly, a 5′ HindIII and a 3′ BamH1 site were added as well as stuffer sequences to inset these sites from the extreme ends of the synthetic fragment. These sites allowed cloning of the encoding nucleic acid into pEGEAM3 and expression from the CMV promoter.

The nucleic acid encoding the above non-immunoglobulin Epo-Thyl carrier binding polypeptide was chemically synthesized as described in Example I, cloned into pEGEAM3 and introduced into mammalian cells by transfection according to methods well known in the art, such as those described in Sambrook et al., supra. Following synthesis in mammalian cells, this protein was expressed and should be highly glycosylated in both the Epo and Thy-1 domains.

### EXAMPLE III

### Non-Immunoglobulin Glucagon-Like Peptide 1-Thy1 Carrier Binding Polypeptides

This Example shows the design and synthesis of a non-immunoglobulin glucagon-like peptide (GLP)-Thy1 carrier binding polypeptide for expression in E. coli cells.

GLP-1 is a 31 amino acid peptide with insulinogenic properties useful for the treatment of type II diabetes. However, the peptide is refractory to therapeutic use due to its short serum half-life.

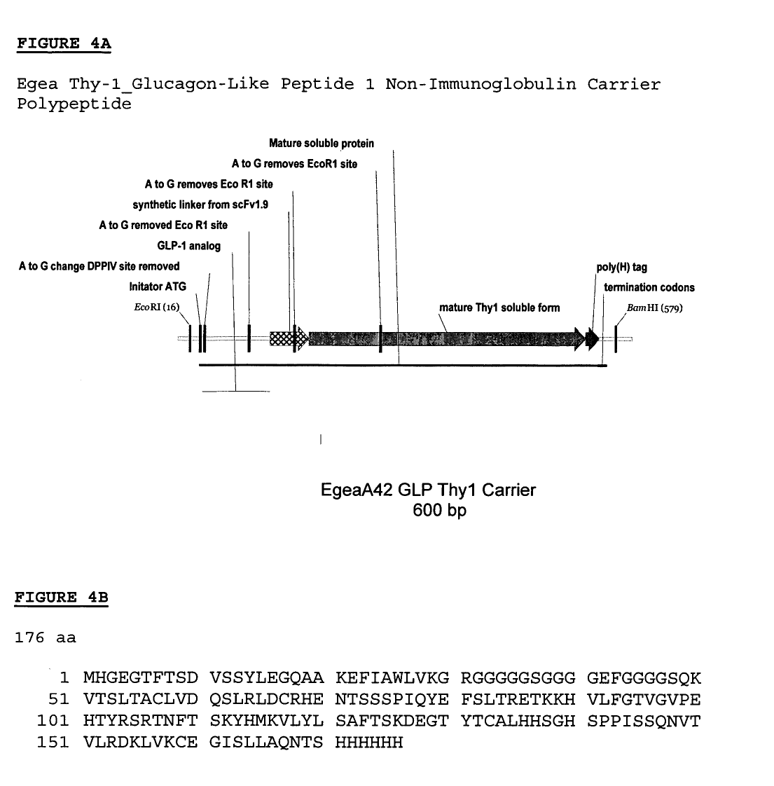

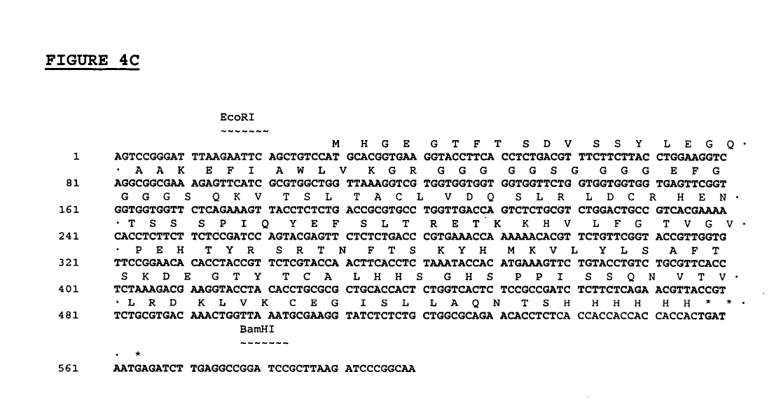

A non-immunoglobulin GLP-Thyl carrier binding polypeptide was designed to consist of a GLP-1 peptide fused to the amino terminus of a Thy1 scaffold polypeptide used as a carrier as described in Example II. A (G₄S)₃-like linker separated the two polypeptide sequences as described in Example II. The leader sequence and Thy1 carrier polypeptide sequence was as described in Example II. A schematic diagram of the resultant chimeric GLP-Thyl carrier binding polypeptide is shown in FIG. 4A.

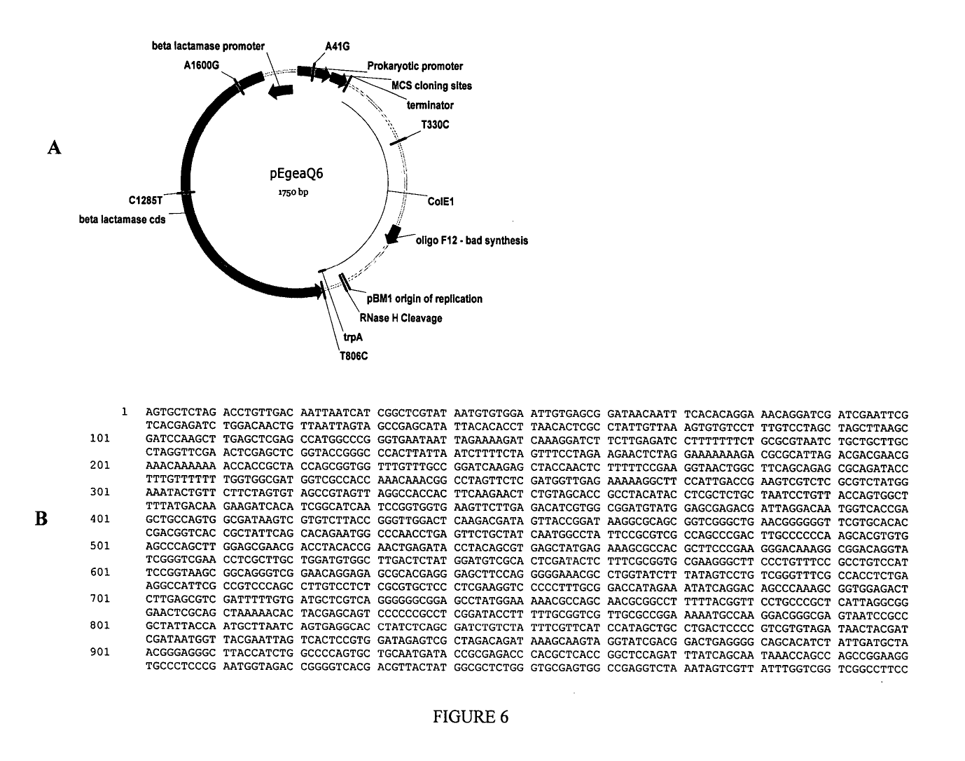

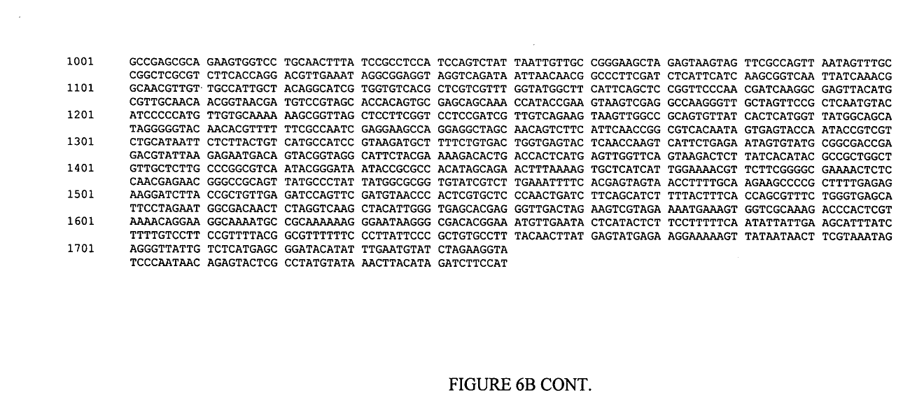

Briefly, the encoding nucleic acid for the above design consists of 600 bp. It was synthesized and incorporated into an expression vector, termed pEgeaQ6, which is the expression vector depicted schematically in FIG. 6. This figure also shows the nucleotide sequence of pEgeaQ6. The amino acid sequence of the chimeric GLP-Thyl carrier binding polypeptide is shown in FIG. 4B. The encoding nucleotide sequence and corresponding amino acid sequence of the GLP-Thyl carrier binding polypeptide is shown in FIG. 4C.

For construction of the non-immunoglobulin GLP-Thyl carrier binding polypeptide, the human GLP-1 peptide amino acid sequence was used as a starting place equivalent to amino acids 98-127 of the mature preproglucagon, amino acids 1-31. The second amino acid residue (A₂) of GLP-1 was replaced by a G₂ from extendin-4, to eliminate the DPP IV peptidase cleavage site (Dipeptidyl aminopeptidase IV). A synthetic linker was incorporated following the GLP sequence that was based on the anti-fibrin single chain antibody (GGGGS)₃ synthetic linker in ScFv1.9.

The Thy1 sequence used was taken from the NCHI database which corresponds to the human preprotein version. The leader sequence and transmembrane regions were removed. All three carbohydrate (CHO) addition sites were retained, as was the entire Ig-like domain of the scaffold polypeptide, which can be modeled based on a single antibody variable region. Finally, an initiator ATG (and initiator Met) was added at the amino terminal of the above encoding nucleic acid.

To produce the encoding nucleotide sequence, gene back-translation was performed using DeanWriter Gold 0.5 and E coli codons. Three substitutions were made to remove three internal EcOR1 sites, all were A to G changes. Further, a 6× poly His (H) was added at the carboxy terminal to aid in protein purification. Three termination codons were also added to the final encoding nucleic acid. Finally, a 5′ EcOR1 and a 3′ BamH1 site were added as well as stuffer sequences to inset these sites from the extreme ends of the synthetic fragment. These sites allowed cloning of the encoding nucleic acid into pEGEAQ6 and expression from the Tac promoter in E. coli cells.

The nucleic acid encoding the above non-immunoglobulin GLP-Thyl carrier binding polypeptide was chemically synthesized as described in Example I, cloned into pEGEAQ6 and introduced into E. coli cells by transfection according to methods well known in the art such as those described in Sambrook et al., supra. Following synthesis in procaryotic cells, this non-immunoglobulin polypeptide can be purified using the His tag and affinity purification procedures.

Throughout this application various publications have been referenced within parentheses or otherwise. The disclosures of these publications, and the references cited therein, in their entireties are hereby incorporated by reference in this application in order to more fully describe the state of the art to which this invention pertains.

Although the invention has been described with reference to the disclosed embodiments, those skilled in the art will readily appreciate that the specific examples and studies detailed above are only illustrative of the invention. It should be understood that various modifications can be made without departing from the spirit of the invention. Accordingly, the invention is limited only by the following claims.

We claim:

1. A chimeric non-immunoglobulin binding polypeptide comprising an immunoglobulin-like domain containing scaffold having two or more solvent exposed loops containing a different CDR from a parent antibody inserted into each of said two or more loops and exhibiting selective binding activity toward a ligand bound by said parent antibody.

2. The chimeric non-immunoglobulin binding polypeptide of claim 1, wherein said immunoglobulin-like domain containing scaffold comprises a ThyOx family polypeptide, or a functional fragment thereof.

3. The chimeric non-immunoglobulin binding polypeptide of claim 2, wherein said ThyOx family polypeptide is selected from the group of Ox2, CD7, Ox2-like protein and Ox2 homolog, or a functional fragment thereof.

4. The chimeric non-immunoglobulin binding polypeptide of claim 2, wherein said ThyOx family polypeptide comprises Thy-1, or a functional fragment thereof.

5. The chimeric non-immunoglobulin binding polypeptide of claim 4, wherein said two or more solvent exposed loops comprise amino acid residues 47-51, 67-98, 67-98 (Δ81-96) or 130-140 of Thy-1.

6. The chimeric non-immunoglobulin binding polypeptide of claim 1, wherein said immunoglobulin-like domain containing scaffold is selected from the group of T cell receptor, CD8, CD4, CD2, class I MHC, class II MHC, CD1, cytokine receptor, GCSF receptor, GMCSF receptor, hormone receptor, growth hormone receptor, erythropoietin receptor, interferon receptor, interferon gamma receptor, prolactin receptor, NCAM, VCAM, ICAM, N-caderin, E-caderin, fibronectin, tenascin, and I-set containing domain polypeptides, or a functional fragment thereof.

7. The chimeric non-immunoglobulin binding polypeptide of claim 1, further comprising at least three solvent exposed loops.

8. The chimeric non-immunoglobulin binding polypeptide of claim 7, further comprising a different CDR from said parent antibody inserted into said at least three solvent exposed loops.

9. The chimeric non-immunoglobulin binding polypeptide of claim 1, wherein said different CDRs from said parent antibody are selected from CDR1, CDR2 and CDR3.

10. A chimeric non-immunoglobulin binding polypeptide comprising an immunoglobulin-like domain containing scaffold having less than about 20% sequence identity to a human immunoglobulin variable region framework domain, said immunoglobulin-like domain containing scaffold having two or more altered solvent exposed loops and exhibiting selective binding activity toward a disparate ligand.

11. The chimeric non-immunoglobulin binding polypeptide of claim 10, wherein said immunoglobulin-like domain containing scaffold comprises a ThyOx family polypeptide, or a functional fragment thereof.

12. The non-immunoglobulin binding polypeptide of claim 11, wherein said ThyOx family polypeptide is selected from the group of Ox2, CD7, Ox2-like protein and Ox2 homolog, or a functional fragment thereof.

13. The chimeric non-immunoglobulin binding polypeptide of claim 11, wherein said ThyOx family polypeptide comprises Thy-1, or a functional fragment thereof.

14. The chimeric non-immunoglobulin binding polypeptide of claim 13, wherein said two or more altered solvent exposed loops comprise amino acid residues 47-51, 67-98, 67-98 (Δ81-96) or 130-140 of Thy-1.

15. The chimeric non-immunoglobulin binding polypeptide of claim 10, wherein said immunoglobulin-like domain containing scaffold is selected from the group of T cell receptor, CD8, CD4, CD2, class I MHC, class II MHC, CD1, cytokine receptor, GCSF receptor, GMCSF receptor, hormone receptor, growth hormone receptor, erythropoietin receptor, interferon receptor, interferon gamma receptor, prolactin receptor, NCAM, VCAM, ICAM, N-caderin, E-caderin, fibronectin, tenascin, and I-set containing domain polypeptides, or a functional fragment thereof.

16. The chimeric non-immunoglobulin binding polypeptide of claim 10, wherein said two or more altered solvent exposed loops further comprise a ligand binding domain from a parent binding polypeptide.

17. The chimeric non-immunoglobulin binding polypeptide of claim 16, wherein said parent binding polypeptide is selected from the group of EPO, 8E5 and GLP.

18. The chimeric non-immunoglobulin binding polypeptide of claim 16, wherein said disparate ligand comprises a ligand bound by said parent polypeptide.

19. The chimeric non-immunoglobulin binding polypeptide of claim 10, wherein said two or more altered solvent exposed loops further comprise different CDR region sequences from a parent antibody.

20. The chimeric non-immunoglobulin binding polypeptide of claim 10, further comprising at least three altered solvent exposed loops.

21. The chimeric non-immunoglobulin binding polypeptide of claim 20, wherein said at least three altered solvent exposed loops further comprise a different CDR region sequence from a parent antibody.

22. The chimeric non-immunoglobulin binding polypeptide of claims 20 or 21, wherein said CDR region sequences from said parent antibody are selected from CDR1, CDR2 and CDR3.

23. A chimeric ThyOx binding polypeptide comprising one or more altered immunoglobulin-like domain loop regions of a ThyOx family polypeptide and having selective binding activity toward a non-ThyOx ligand.

24. The chimeric ThyOx binding polypeptide of claim 23, wherein said ThyOx family polypeptide is selected from the group of Ox2, CD7, Ox2-like protein and Ox2 homolog, or a functional fragment thereof.

25. The chimeric ThyOx binding polypeptide of claim 23, wherein said ThyOx family polypeptide comprises Thy-1, or a functional fragment thereof.

26. The chimeric ThyOx binding polypeptide of claim 25, wherein said one or more altered immunoglobulin-like domain loop regions comprise amino acid residues 47-51, 67-98, 67-98 (Δ81-96) or 130-140 of Thy-1.

27. The chimeric ThyOx binding polypeptide of claim 23, wherein said one or more altered immunoglobulin-like domain loop regions further comprise a ligand binding domain from a parent binding polypeptide.

28. The chimeric ThyOx binding polypeptide of claim 27, wherein said parent binding polypeptide is selected from the group of EPO, 8E5 and GLP.

29. The chimeric ThyOx binding polypeptide of claim 27, wherein said non-ThyOx ligand comprises a ligand bound by said parent polypeptide.

30. The chimeric ThyOx binding polypeptide of claim 23, wherein said one or more altered immunoglobulin-like domain loop regions further comprise different CDR region sequences from a parent antibody.

31. The chimeric ThyOx binding polypeptide of claim 23, further comprising at least three altered immunoglobulin-like domain loop regionss.

32. The chimeric ThyOx binding polypeptide of claim 31, wherein said at least three altered immunoglobulin-like loop regions further comprise a different CDR region sequence from a parent antibody.

33. The chimeric ThyOx binding polypeptide of claims 30 or 32, wherein said CDR region sequences from said parent antibody are selected from CDR1, CDR2 and CDR3.

34. The chimeric ThyOx binding polypeptide of claim 33, wherein said non-ThyOx ligand comprises a ligand bound by said parent antibody.

35. A chimeric ThyOx carrier polypeptide comprising a at least one immunoglobulin-like domain containing scaffold derived from a ThyOx family polypeptide, and a heterologous binding polypeptide exhibiting selective binding activity toward a non-ThyOx ligand.

36. The chimeric ThyOx binding polypeptide of claim 35, wherein said ThyOx family polypeptide is selected from the group of Ox2, DD7, Ox2-like protein and Ox2 homolog, or a functional fragment thereof.

37. The chimeric ThyOx binding polypeptide of claim 35, wherein said ThyOx family polypeptide comprises Thy-1, or a functional fragment thereof.

38. The chimeric ThyOx binding polypeptide of claim 35, wherein said heterologous binding polypeptide comprises glucagon-like peptide, erythropoietin, an antibody variable region, or a functional fragment thereof.

39. The chimeric ThyOx binding polypeptide of claim 35, wherein said non-ThyOx ligand comprises a ligand bound by said heterologous binding polypeptide.

40. A nucleic acid encoding a non-immunoglobulin or ThyOx binding polypeptide of claims 1, 10, 23 or 35.


SEQUENCE LISTING (13 sequences)

SEQ ID NO: 1 - protein, 162 aa, Homo sapiens
MNLAISIALLLTVLQVSRGQKVTSLTACLVDQSLRLDCRHENTSSSPIQYEESLTRETKKHVLFGTVGVPEHTYRSRTNFTSKYHMKVLYLSAFTSKDEGTYTCALHHSGHSPPILSSQNVTVLRDKLVKCEGISLLAQNTSWLLLLLLSLSLLQATDFMSL

SEQ ID NO: 2 - protein, 124 aa, Homo sapiens
QLQQSGEALVKPGASVRMSCKASGYTFPNYWMHWVKQRPGQGLEWIGTIDPADSYTSYNQNFKDKATLTVKPSSTAYMQLSSLTFGDSAVYFCARESYYYRYYFDYWGHGTTLTVSSAKTTPKL

SEQ ID NO: 3 - protein, 37 aa, Artificial Sequence
  Consensus peptide
QLSLKLCKSSFRGTIDNFKDATTSISSEGIWLSLSTL

SEQ ID NO: 4 - protein, 111 aa, Artificial Sequence
  ThyOx non-immunoglobulin binding polypeptide
QVSRGQKVTSLTACLVDQSLRLDCRHENTSSSNYWMHFSLTRETKKHVLFGTIDPADSYTSYNQNFKDEGTYTCALHHSGHSPPISSQNVTVLRDKLVKCEGVYYRYYFDY

SEQ ID NO: 5 - DNA, 1050 bp, Artificial Sequence
  carrier encoding erytropoietin
gattggcgaagcttggaggaatgggcgtgcacgagtgccccgcctggctgtggctgctgctgagcctgctgagcctgcccctgggcctgcccgtgctgggcgcccccccccggctgatctgcgacagccgggtgctggagcggcacctgctggaggccaaggaggccgagagcatcaccaccggctgcgtggaggactgcagcctgaacgagaacatcaccgtgcccgacagcaaggtgaacttctacgcctggaagcggatggaggtgggccagcaggccgtggaggtgtggcagggcctggccctgctgagcgaggccgtgctgcggggccaggccctgctggtgatcagcagccagccctgggagcccctgcagctgcacgtggacaaggccgtgagcggcctgcggagcctgaccaccctgctgcgggccctgggcgcccagaaggaggccatcagcccccccgacgccgccagcgccgcccccctgcggaccatcaccgccgacaccttccggaagctgttccgggtgtaccccaacttcctgcggggcaagctgaagttctacaccggcgaggcctgccggggcggcggcggcggcagcggcggcggcggcgagttcggcggcggcggcagccagaaggtgaccagcctgaccgcctgcctggtggaccagagcctgcggctggactgccggcacgagaacaccagcagcagccccatccagtacgagttcagcctgacccgggagaccaagaagcacgtgctgttcggcaccgtgggcgtgcccgagcacacctaccggagccggaccaacttcaccagcaagtaccacatgaaggtgctgtacctgagcgccttcaccagcaaggacgagggcacctacacctgcgccctgcaccacagcggccacagcccccccatcagcagccagaacgtgaccgtgctgcgggacaagctggtgaagtgcgagggcatcagcctgctggcccagaacaccagccaccaccaccaccaccactgatgataagatcggatcctaggcttcc

SEQ ID NO: 6 - protein, 334 aa, Artificial Sequence
  chimeric ThyOx carrier polypeptide containing erythropoietin
MGVHECPAWLWLLLSLLSLPLGLPVLGAPPRLICDSRVLERHLLEAKEAESITTGCVEDCSLNENITVPDSKVNFYAWKRMEVGQQAVEVWQGLALLSEAVLRGQALLVISSQPWEPLQLHVDKAVSGLRSLTTLLRALGAQKEAISPPDAASAAPLRTITADTFRKLFRVYPNFLRGKLKFYTGEACRGGGGGSGGGGEFGGGGSQKVTSLTACLVDQSLRLDCRHENTSSSPIQYEFSLTRETKKHVLFGTVGVPEHTYRSRTNFTSKYHMKVLYLSAFTSKDEGTYTCALHHSGHSPPISSQNVTVLRDKLVKCEGISLLAQNTSHHHHHH

SEQ ID NO: 7 - DNA, 1050 bp, Artificial Sequence; identical to SEQ ID NO: 5
  SuperEpo

SEQ ID NO: 8 - protein, 334 aa, Artificial Sequence; identical to SEQ ID NO: 6
  SuperEpo

SEQ ID NO: 9 - DNA, 600 bp, Artificial Sequence
  carrier encoding glucagon-like peptide 1
agtccgggatttaagaattcagctgtccatgcacggtgaaggtaccttcacctctgacgtttcttcttacctggaaggtcaggcggcgaaagagttcatcgcgtggctggttaaaggtcgtggtggtggtggtggttctggtggtggtggtgagttcggtggtggtggttctcagaaagttacctctctgaccgcgtgcctggttgaccagtctctgcgtctggactgccgtcacgaaaacacctcttcttctccgatccagtacgagttctctctgacccgtgaaaccaaaaaacacgttctgttcggtaccgttggtgttccggaacacacctaccgttctcgtaccaacttcacctctaaataccacatgaaagttctgtacctgtctgcgttcacctctaaagacgaaggtacctacacctgcgcgctgcaccactctggtcactctccgccgatctcttctcagaacgttaccgttctgcgtgacaaactggttaaatgcgaaggtatctctctgctggcgcagaacacctctcaccaccaccaccaccactgataatgagatcttgaggccggatccgcttaagatcccggcaa

SEQ ID NO: 10 - protein, 176 aa, Artificial Sequence
  chimeric ThyOx carrier polypeptide containing glucagon-like peptide 1
MHGEGTFTSDVSSYLEGQAAKEFIAWLVKGRGGGGGSGGGGEFGGGGSQKVTSLTACLVDQSLRLDCRHENTSSSPIQYEFSLTRETKKHVLFGTVGVPEHTYRSRTNFTSKYHMKVLYLSAFTSKDEGTYTCALHHSGHSPPISSQNVTVLRDKLVKCEGISLLAQNTSHHHHHH

SEQ ID NO: 11 - DNA, 4000 bp, Artificial Sequence
  vector pEgea M3
gattattctagacccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagaatcgaaattaatacgactcactatagggagacccaagctggctagcgtttaaacttaagcttggtaccgagctcggatccactctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggacgcaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctaatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaaccagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatctagaggttattgtct

SEQ ID NO: 12 - DNA, 1750 bp, Artificial Sequence
  vector pEgea Q6
agtgctctagacctgttgacaattaatcatcggctcgtataatgtgtggaattgtgagcggataacaatttcacacaggaaacaggatcgatcgaattcggatccaagcttgagctcgagccatggcccgggtgaataattagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggacaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgcccgctcattaggcgggctattaccaatgcttaatcagtgaggcacctatctcagcgatctgtctatttcgttcatccatagctgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgctcccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatctagaaggta

SEQ ID NO: 13 - DNA, 15 bp, Artificial Sequence
  synthetic linker
ggggsggggsggggs